(19) United States
(12) Patent Application Publication
(10) Pub. No.: US 2017/0202217 A1
(43) Pub. Date: Jul. 20, 2017

(54) ANTIBACTERIAL POLYMER, PRODUCTION METHOD THEREFOR, AND USAGE THEREOF

(71) Applicant: NATIONAL UNIVERSITY CORPORATION YAMAGATA UNIVERSITY, Yamagata (JP)

(72) Inventors: Masaru Tanaka, Yamagata (JP); Kazuki Fukushima, Yamagata (JP); Chikako Sato, Yamagata (JP); Ayano Sasaki, Yamagata (JP); Kouhei Kishi, Yamagata (JP)

(73) Assignee: NATIONAL UNIVERSITY CORPORATION YAMAGATA UNIVERSITY, Yamagata (JP)

(21) Appl. No.: 15/309,722

(22) PCT Filed: May 8, 2015
(86) PCT No.: PCT/JP2015/063380

§ 371 Date: Mar. 28, 2017

(30) Foreign Application Priority Data
May 8, 2014 (JP) 2014-097237

(51) Int. Cl. A01N 33/12; A61L 31/16; A61L 31/10; C08G 64/42; C08F 220/34

(52) U.S. Cl. CPC A01N 33/12; C08G 64/42; C08F 220/34; A61L 31/10; A61L 31/16; A61L 2300/404

(57) ABSTRACT

Provided is a polymer material having effective antibacterial properties and having bio adaptability in which cytotoxicity, especially low hemolysis, when used in contact with in vivo tissue and blood is suppressed so as to be low. The polymer is characterized by comprising a main chain and a side chain portion which is linked to the main chain via a linker and which contains at least a structure specified by the following (A) and (B). (A) A structure containing a cationic group and (B) a structure in which expression of bio adaptability is expected.

### TECHNICAL FIELD

The present invention relates to an antibacterial polymer, and more particularly, to a polymer that demonstrates biocompatibility when used in contact with biological tissue or blood.

### BACKGROUND ART

Although the proliferation of antibiotics has significantly improved the suffering caused by infections, the appearance of multiple-drug-resistant bacteria due to the overuse of antibiotics has resulted in new problems that are difficult to resolve with existing antibiotics. In addition, patients who have been administered immunosuppressants for organ transplant surgery and the like as well as elderly persons whose immune function has decreased due to aging are susceptible to microbial infections attributable to artificial heart valves, catheters and other artificial materials implanted in the body. Cationic polymers are attracting attention as a novel material for preventing infections in such cases. Since nearly all conventional antibiotics target a specific protein or other substance in cells, the mutation of these target biomolecules makes it possible for bacteria to easily acquire resistance. In contrast, since cationic peptides such as magainin or cecropin impair interaction with the cell membrane of bacteria based on an amphiphilic structure having a positive charge, it is possible to demonstrate antibacterial activity that suppresses the appearance of resistant bacteria.

On the other hand, these naturally-occurring antibacterial peptides are limited to specific pharmaceutical applications due to their high production cost and difficulty in mass production. Therefore, amphiphilic arylamide polymers have been reported that can be prepared from inexpensive monomers and mimic the physical and biological properties of these antibacterial peptides (see Non-Patent Document 1). Among these, the polymer having the highest level of antibacterial activity has a minimum inhibitory concentration (MIC) of 50 μg/mL or less against pathogenic bacteria such as Escherichia coli, Salmonella species or Pseudomonas aeruginosa (see Table 2 of Non-Patent Document 1).

In addition, there is also a report describing the results of investigating antibacterial activity and hemolytic activity for cationic amphiphilic polymethacrylate derivatives having various molecular weights prepared by radially polymerizing N-(t-butoxycarbonyl)aminoethyl methacrylate and butyl methacrylate (see Non-Patent Document 2). According to those results, low molecular weight polymers having a molecular weight of 2000 or less exhibited the lowest MIC values and demonstrated reduced hemolytic activity in comparison with high molecular weight polymers. In addition, selective antibacterial activity with respect to hemolytic activity decreased as the content of butyl groups increased.

Moreover, Patent Document 1 describes a biodegradable cationic block copolymer prepared by ring-opening polymerization and a method for using the same in antibiotic applications. Amphiphilic block copolymers, which contain a cationic hydrophilic block and a hydrophobic block, form nanostructures in aqueous solution, which are thought to result in increases in cationic charge and local concentrations of polymeric substances, enhance interaction with the negatively charged cell wall, and eventually bring about more potent antimicrobial activity.

### PRIOR ART DOCUMENTS

### Non-Patent Documents

Non-Patent Document 1: G. N. Tew, et al, PNAS 2002, Vol. 99, pp. 5110-5114

Non-Patent Document 2: K. Kuroda and W. F. DeGrado, J. Am. Chem. Soc., 2005, 127, 4128-4129

### Patent Documents

Patent Document 1: Japanese Translation of PCT International Application Publication No. 2013-515815

### DISCLOSURE OF THE INVENTION

### Problems to be Solved by the Invention

In general, artificially synthesized polymers that mimic the physical and biological properties of antibacterial peptides are known to demonstrate a decrease in blood compatibility, and exhibit an increase in hemolysis caused by destruction of erythrocytes in particular, in the case of having increased antibacterial activity against Gram negative bacteria such as highly pathogenic Escherichia coli. In addition, when the safety of these polymers with respect to the blood and body is increased, there are many cases in which antibacterial activity against Gram negative bacteria decreases or is lost, thus making the realization of both antibacterial activity and blood compatibility a frequently encountered problem in the development of antibacterial materials.

Therefore, an object of the present invention is to provide a polymer material, which in addition to having effective antibacterial activity, demonstrates biocompatibility in which cytotoxicity, and particularly hemolytic activity, is held to a low level when used in contact with biological tissue or blood.

### Means for Solving the Problems

The present invention has the characteristics indicated below in order to solve the aforementioned problems.

(1) A polymer constituted with a main chain and side chain moieties at least containing structures specified by the following (A) and (B) which are linked via a linker:

(A) a structure containing a cationic group; and,

(B) a structure expected to express biocompatibility.

(2) The polymer described in (1), wherein the cationic group is a primary to quaternary ammonium group.

(3) The polymer described in (1), wherein the cationic group is a primary ammonium group.

(4) The polymer described in any of (1) to (3), wherein the structure expected to express biocompatibility contains at least one ether structure.

(5) The polymer described in (4), wherein the ether structure is a linear ether structure or cyclic ether structure.

(6) The polymer described in any of (1) to (5), wherein the main chain is a biodegradable polymer, a non-biodegradable polymer or a copolymer thereof.

(7) The polymer described in any of (1) to (6), wherein the main chain is a biodegradable polymer.

(8) The polymer described in any of (1) to (7), which is a polycarbonate having a side chain moiety containing a primary ammonium group linked via a linker, and a side chain moiety containing a chain ether structure linked via a linker.

(9) The polymer described in any of (1) to (8), wherein the linker is an ester bond.

(10) The polymer described in any of (1) to (6), (8) and (9), wherein the main chain is a polycarbonate chain or hydrocarbon chain.

(11) The polymer described in any of (1) to (10), which is used in contact with biological tissue or blood.

(12) A method for producing a polymer, comprising a step of ring-opening polymerization at least two types of monomer compounds selected from cyclic monomers represented by general formula (I):

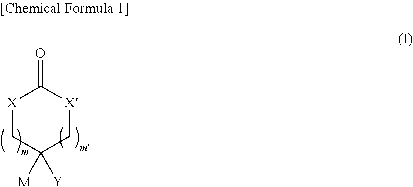

(wherein,

X and X′ mutually and independently represent —O—, —NH— or CH₂— provided that at least one thereof is not —CH₂—,

Y represents a group represented by L-Z (wherein, Z represents a side chain moiety having a cationic group, a side chain moiety Z¹ having a group serving as a precursor of a cationic group, or a side chain moiety Z² capable of retaining intermediate water in the body and L represents a linker between the main chain and Z that is selected from unit structures having an alkylene group, ether bond, thioether bond, ester bond, amide bond, urethane bond, urea bond or a combination thereof),

M represents a hydrogen atom or a linear or branched alkyl group having 3 carbon atoms or less, and

m and m′ mutually and independently represent an integer of 0 to 5, provided that at least one of m and m′ is not zero when X and X′ are both —O—, and the sum of m and m′ is 7 or less); wherein, a first cyclic monomer has Z¹ and a second cyclic monomer has Z².

(13) The production method described in (12), wherein the mixing ratio between the first cyclic monomer and the second cyclic monomer is 1:99 to 99:1 in terms of the molar ratio thereof.

(14) A medical device having the polymer described in any of (1) to (11) on at least a portion of the surface thereof.

(15) An antibacterial agent comprising the polymer described in any of (1) to (11).

### Effects of the Invention

The polymer of the present invention demonstrates superior antibacterial action at a low concentration in addition to demonstrating superior biocompatibility. The polymer of the present invention can be used in infection countermeasures in various health care settings as a result of being applicable to various processing treatment such as that for preparing a liquid, solid or coating agent.

### BRIEF DESCRIPTION OF THE DRAWINGS

FIG. 1 shows the ¹H-NMR spectrum (400 MHz, CDCl₃) of a Compound (2) synthesized in Example 1.

FIG. 2 shows the ¹H-NMR spectrum (400 MHz, CDCl₃) of a Compound (3) synthesized in Example 2.

FIG. 3 shows the ¹H-NMR spectrum (400 MHz, acetone-d₆) of a Compound (4) synthesized in Example 3.

FIG. 4 shows the ¹H-NMR spectrum (400 MHz, acetone-d₆) of a Compound (5) synthesized in Example 4.

FIG. 5 shows the ¹H-NMR spectrum (400 MHz, acetone-d₆) of a Compound (6) synthesized in Example 5.

FIG. 6 shows the ¹H-NMR spectrum (400 MHz, acetone-d₆) of a Compound (7) synthesized in Example 5.

FIG. 7 shows the ¹H-NMR spectrum (500 MHz, CDCl₃) of a Polymer (8) synthesized in Example 6.

FIG. 8 shows the ¹H-NMR spectrum (400 MHz, DMSO-d₆) of a Polymer (10) synthesized in Example 7.

FIG. 9 shows the ¹H-NMR spectrum (500 MHz, DMSO-d₆) of a Polymer (11) synthesized in Example 11.

FIG. 10 shows the ¹H-NMR spectrum (500 MHz, DMSO-d₆) of a Polymer (12) synthesized in Example 13.

FIG. 11 shows the ¹H-NMR spectrum (400 MHz, MeOH-d₄) of a Polymer (13) synthesized in Example 14.

FIG. 12 shows the ¹H-NMR spectrum (400 MHz, MeOH-d₄) of a Polymer (14) synthesized in Example 15.

FIG. 13 shows the ¹H-NMR spectrum (500 MHz, MeOH-d₄) of a Polymer (15) synthesized in Example 16.

FIG. 14 shows the ¹H-NMR spectrum (500 MHz, MeOH-d₄) of a Polymer (16) synthesized in Example 17.

FIG. 15 shows time-based changes in the growth behavior of Escherichia coli treated with various concentrations of a Polymer (11) (MIC=16 mg/l).

FIG. 16 shows time-based changes in the growth behavior of Escherichia coli treated with various concentrations of a Polymer (12) (MIC=16 mg/l).

FIG. 17 shows time-based changes in the growth behavior of Escherichia coli treated with various concentrations of a Polymer (15) (MIC=64 mg/l).

FIG. 18 shows time-based changes in the growth behavior of Escherichia coli treated with various concentrations of a Polymer (16) (MIC=32 mg/l).

FIG. 19 shows time-based changes in the growth behavior of Escherichia coli cultured in various concentrations of PEI (MIC=250 mg/l).

FIG. 20 shows time-based changes in the growth behavior of Escherichia coli cultured in various concentrations of PEG (no MIC).

FIG. 21 shows time-based changes in the growth behavior of Escherichia coli cultured in various concentrations of P/S (MIC=4 mg/l).

FIG. 22 shows SEM images of Escherichia coli treated with various polymers (32 mg/l).

FIG. 23 indicates the results of a hemolysis test on various polymers.

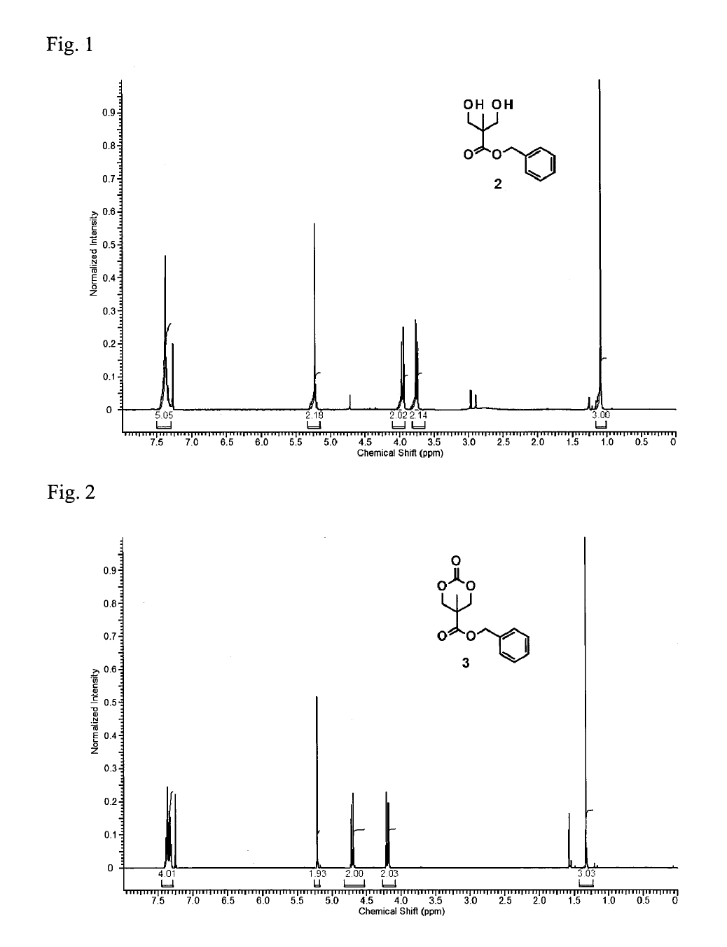

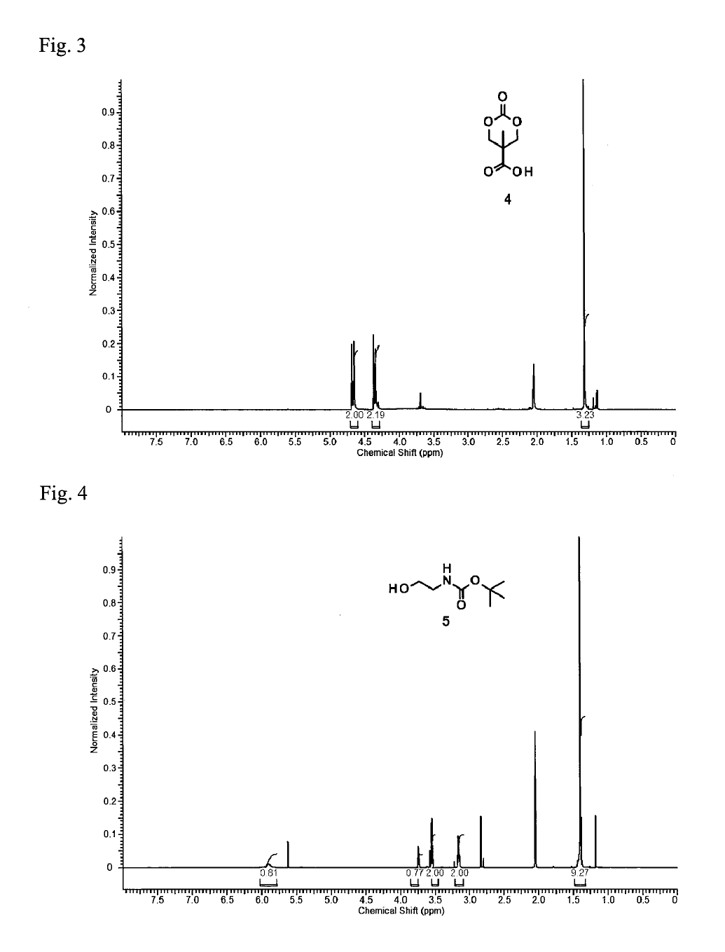

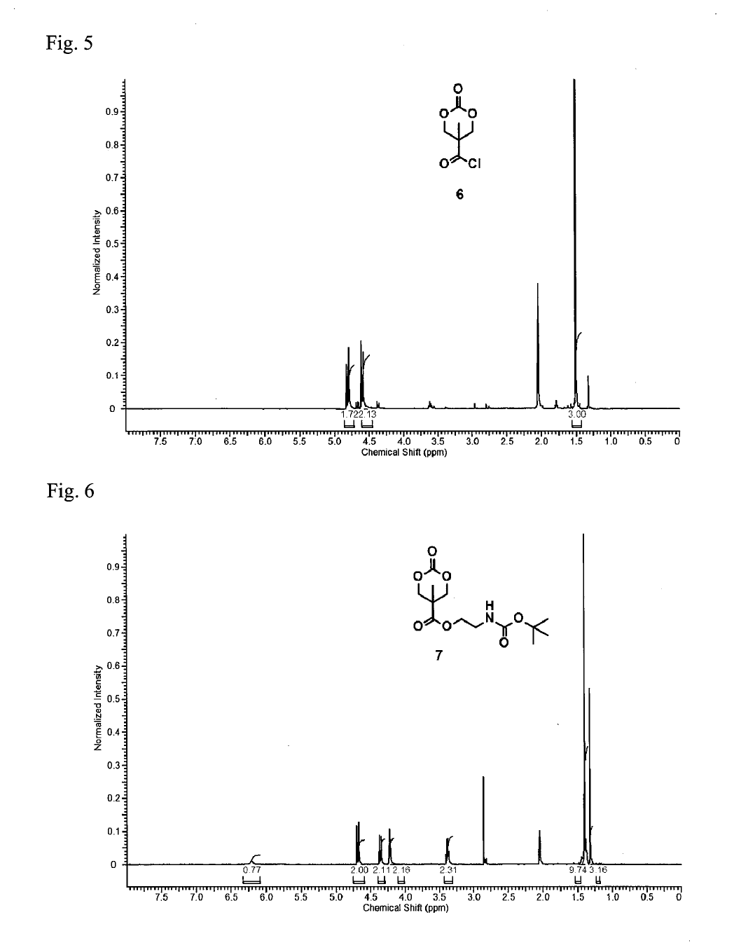

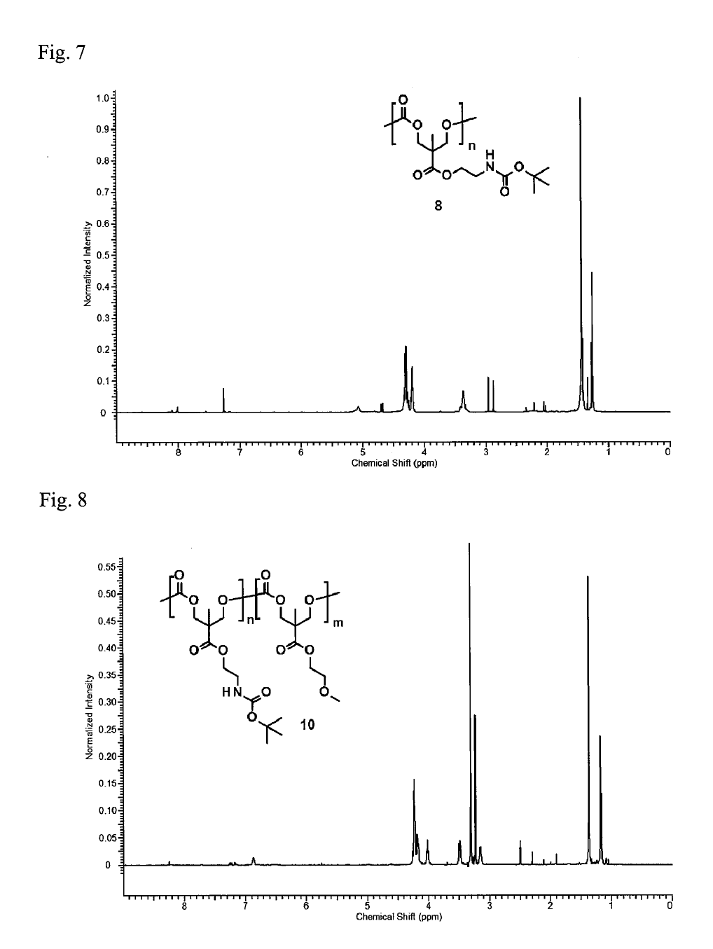

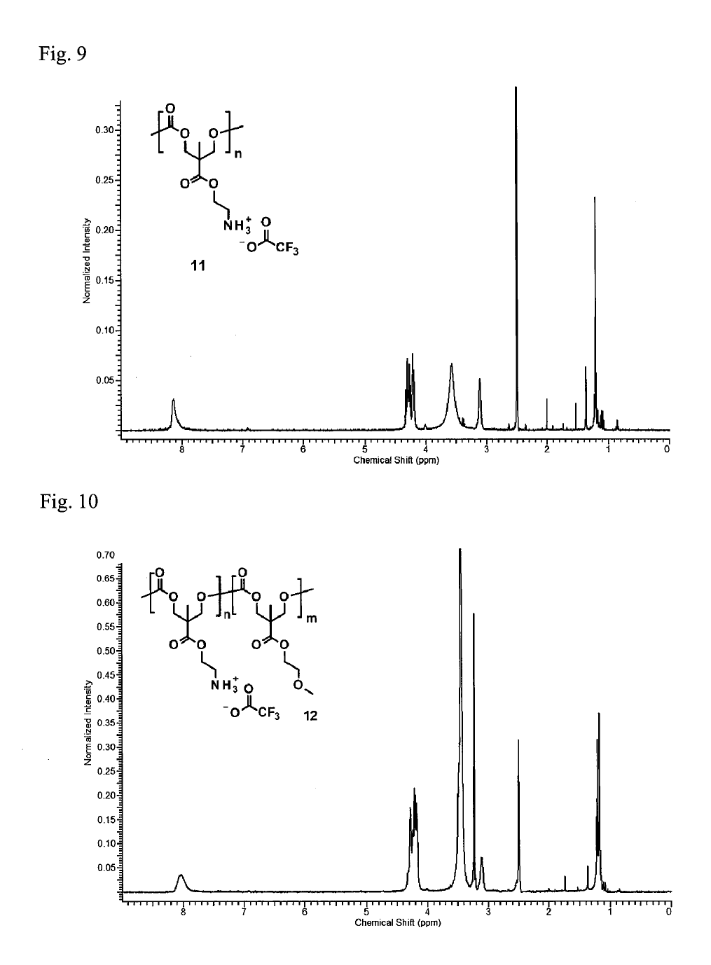

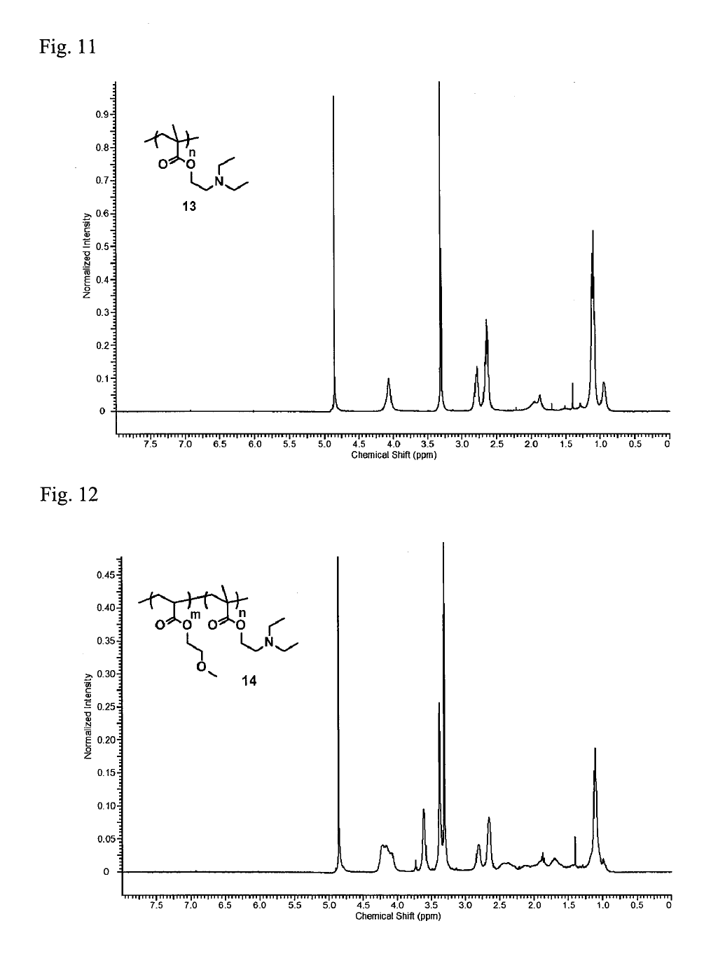

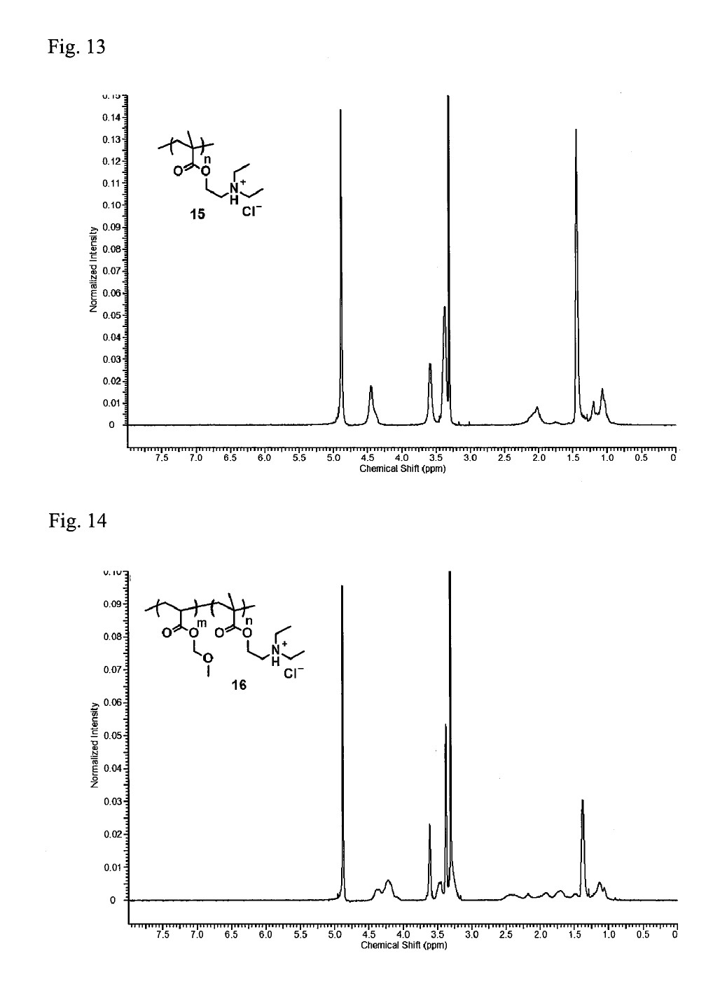

### BEST MODE FOR CARRYING OUT THE INVENTION

The present invention is based on the finding that, in a polymer comprising a side chain containing a cationic group expected to demonstrate antibacterial activity and a side chain containing a structure able to demonstrate biocompatibility at a suitable ratio thereof, antibacterial activity can be demonstrated while demonstrating prescribed biocompatibility. In other words, a mechanism is presumed to exist whereby, when the polymer has contacted biological material (cells) in the manner of Escherichia coli or human erythrocytes, although the structure of the cationic group has an effect that acts towards destruction of cells, the structure demonstrating biocompatibility inhibits that effect, the mechanism by which each structure generates an effect on biological material is present at similar levels, and a relationship is thought to exist in which both act on biological material in mutual competition. As a result of using this finding, the degree of the effects on biological material can be adjusted with a polymer incorporating both structures at a prescribed ratio, thereby clearly making it possible to selectively accommodate various types of biological materials.

The following provides a detailed explanation of the present invention.

### Definition of Terms

In the present invention, the following terms are used to indicate respectively explained contents regardless of whether they appear alone or appear in combination.

In the present description, the term “alkyl group” indicates a monovalent saturated hydrocarbon group containing a linear or branched carbon chain having a skeleton composed of carbon atoms. In addition, the term “alkylene group” indicates a divalent hydrocarbon group composed of a linear carbon chain. The term “alkylene oxide chain” indicates a structure in which carbon atoms other than on the terminals of the alkylene group are substituted with ether bonds. A “lower alkyl group” or “lower alkylene group” indicates the aforementioned alkyl or alkylene group in which the number of carbon atoms is within the range of 1 to 6.

The term “alkenyl” indicates a monovalent saturated hydrocarbon group containing a linear or branched carbon chain having one or more carbon-carbon double bonds in a skeleton composed of carbon atoms. Although there are no particular limitations thereon, the number of carbon atoms of the alkenyl group is preferably 2 to 20 carbon atoms, more preferably 2 to 10 carbon atoms and most preferably 2 to 6 carbon atoms. Examples of alkenyl groups include, but are not limited to, ethenyl (vinyl), propenyl, butenyl, 2-methylpropenyl, pentenyl and hexenyl groups. In addition, the term “alkynyl” indicates a monovalent saturated hydrocarbon group containing a linear or branched carbon chain having one or more carbon-carbon triple bonds in a skeleton composed of carbon atoms. Although there are no particular limitations thereon, the number of carbon atoms of the alkenyl group is preferably 2 to 20 carbon atoms, more preferably 2 to 10 carbon atoms, and most preferably 2 to 6 carbon atoms. Examples of alkynyl groups include, but are not limited to, ethynyl, propynyl, butynyl, 2-methylpropynyl, pentynyl and hexynyl groups.

In the present description, the term “alkoxy” indicates a monovalent saturated hydrocarbon group in which the aforementioned alkyl group is bound to another molecular structure by an oxygen atom in a structure in which it is bound to an oxygen atom. Although there are no particular limitations thereon, the number of carbon atoms of the alkoxy group is preferably 1 to 20, more preferably 1 to 10 and most preferably 1 to 6. Examples of alkoxy groups include, but are not limited to, methoxy, ethoxy, propoxy, i-propoxy, n-butoxy, i-butoxy, tert-butoxy, pentoxy and hexoxy groups.

The term “alicyclic alkyl” indicates a monovalent aliphatic cyclic hydrocarbon group in which a skeleton composed of carbon atoms forms a ring. Alicyclic alkyl groups are expressed according to the number of carbon atoms that form the ring, and for example, the term “C₃₋₈ alicyclic alkyl” indicates that the number of carbon atoms that form the ring is 3 to 8. Examples of alicyclic alkyl groups include, but are not limited to, cyclopropyl (C₃H₅), cyclobutyl (C₄H₇), cyclopentyl (C₅H₉), cyclohexyl (C₆H₁₁), cycloheptyl (C₇H₁₃) and cyclooctyl (C₈H₁₅) groups.

The term “linear ether” or “alkylene oxide” can be used interchangeably, and indicates a structure in which one —CH₂— moiety other than those on the terminals of the aforementioned alkyl group has been substituted with an ether bond (—O—). In addition, the term “cyclic ether” indicates a structure in which one —CH₂— moiety of the aforementioned alicyclic alkyl group has been substituted with an ether bond.

The term “aryl group” indicates an aromatic substituent containing a single ring or two or three condensed rings. The aryl group preferably contains 6 to 18 carbon atoms, and examples thereof include phenyl, naphthyl, anthracenyl, fluorenyl and indanyl groups.

The term “monomer” or “monomer unit” can be used interchangeably, and refers to a low molecular weight molecule that can be become a constituent of the basic structure of a polymer. Monomers normally have a functional group that serves as the reactive site of a polymerization reaction in the manner of, for example, a carbon-carbon double bond or ester bond.

The term “polymer” or “polymerization product” can be used interchangeably, and refers to a molecule having a structure composed of repeating monomer units that can be obtained from monomers having a low molecular weight. The term “high molecular weight molecule” refers to a polymer as well as a macromolecule obtained by, for example, covalently bonding a large number of atoms in the manner of proteins and nucleic acids.

In the case of a polymer, the term “average degree of polymerization” refers to the average number of monomer units contained in a single polymer molecule. Namely, in a polymer composition, polymer molecules of different lengths are present dispersed over a certain range.

With respect to the molecular weight of a polymer, “number-average molecular weight” refers to the average molecular weight per molecule in a polymer composition, while “weight-average molecular weight” refers to the molecular weight as calculated based on weight. In addition, the ratio between number-average molecular weight and weight-average molecular weight is referred to as the degree of dispersion, and is an indicator of the molecular weight distribution of a polymer composition. The degree of dispersion approaches the average molecular weight of a polymer composition as the value thereof approaches 1, and indicates that a large number of polymer chains of about the same length are contained therein.

In the present invention, the term “biocompatible material” refers to a material that is unlikely to be recognized as a foreign substance when it has contacted a biological material. There are no particular limitations on the biocompatible material provided it is a material that does not exhibit complement activity, thrombotic activity or tissue invasiveness and the like, and includes materials that demonstrate activity so as to induce or not induce specific protein adsorption or cell adhesion. In the present invention, the term “blood-compatible material” refers to the aforementioned biocompatible material that does not cause blood coagulation primarily attributable to platelet attachment or activation.

A phenomenon is known in which, even in the case of an artificially synthesized polymer and the like, a prescribed polymer and the like is unlikely to be recognized as a foreign substance when contacting a biological material. Although the mechanism by which a substance in the form of a foreign substance is inherently not recognized as a foreign substance is not necessarily clear, the inventors of the present invention determined that a special hydration structure referred to as “intermediate water” is present in common on the surfaces of such substances. Since this hydration structure is also observed in common in biological substances, when various types of substances make contact with a biological material, there is clearly a mechanism by which the effects of the various types of substances on the biological material are alleviated or eliminated due to intervention by this hydration structure.

In the present invention, a “biodegradable polymer” refers to a polymer that can be chemically degraded by the action of hydrolysis, oxygen decomposition or microbial degradation and the like. Examples of biodegradable polymers include chemically synthesized polymers in the manner of polyesters or polycarbonates such as polylactic acid or polycaprolactone, biological polymers such as polypeptides, polysaccharides or cellulose, and combinations thereof.

In the present invention, a “side chain” indicates a structure bonded to a polymer main chain that has branched off therefrom.

In addition, in the present invention, an “antibacterial agent” refers to a substance capable of eradicating or inhibiting the growth of bacteria, yeasts, fungi, viruses and protozoans that has antibacterial action in the broad sense as required in medical applications and the like.

In one embodiment of the present invention, a polymer is provided that comprises a main chain and at least two types of side chains via a linker. There are no particular limitations on the main chain that composes the polymer of the present invention, and may have a hydrophobic skeleton such as an alkyl chain. In the case the main chain consists of an alkyl chain, since the polymer is imparted with resistance to water solubility, it can be used to obtain a polymer suitable for applications in the form of a structural material or coating requiring water resistance (water insolubility).

On the other hand, in the case of using a hydrophilic main chain in the manner of polyoxyethylene groups that compose PEG, since water solubility is imparted to the entire polymer, it can be used to obtain a polymer suitable for applications in which it used as a pharmaceutical agent administered into the body in the form of a solution.

In addition, the residence time of a polymer in the body can be adjusted according to whether or not the polymer has biodegradability or the degree of that biodegradability. When the polymer of the present invention is administered into the blood in the manner of an antibiotic or when used in the body as a medical material, the polymer is preferably degraded and absorbed after an amount of time has elapsed that corresponds to the application thereof. Numerous biodegradable polymer structures are commonly known, and these biodegradable polymer structures can be used in the main chain moiety of the polymer of the present invention within a range that is not counter to the object of the present invention. Biodegradable polymers containing a unit structure having an ether bond, thioether bond, ester bond, carbonate group, amide group, urethane bond, urea bond or combination thereof are preferable from the viewpoints of ease of synthesis and diversity in linking with side chains. Non-limiting specific examples thereof include the unit structures indicated in Scheme 1 below.

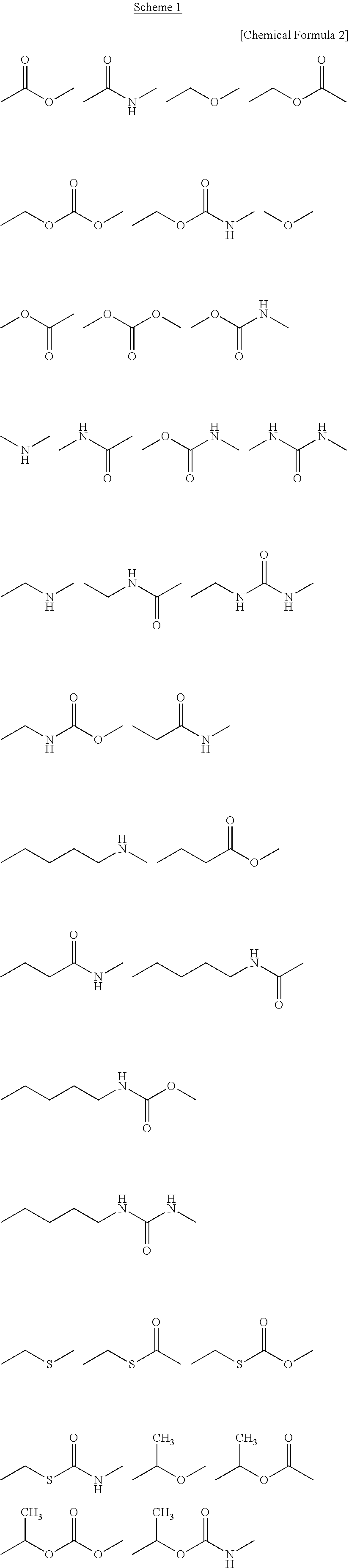

These unit structures that impart biodegradability can contain C₁₋₈ alkylene groups in order to link with the at least two types of side chain moieties according to the present invention, and depending on the case, at least one carbon atom in the C₁₋₈ alkylene group other than carbon atoms adjacent to the aforementioned unit structure may be substituted with a heteroatom selected from N, O or S, and/or a hydrogen atom in the C₁₋₈ alkylene group may be substituted with a lower alkyl group. Side chain moieties are linked to the main chain via a linker. Although there are no particular limitations on the structure of the linker, it is preferably selected from unit structures having an alkylene group, ether bond, thioether bond, ester bond, amide bond, urethane bond, urea bond or combination thereof in order to allow the side chain moieties to efficiently demonstrate antibacterial activity and biocompatibility.

In one embodiment of the present invention, the aforementioned main chain can be in the form of a copolymer of a biodegradable polymer and a non-biodegradable polymer. A polymer commonly known to be a non-biodegradable polymer among persons with ordinary skill in the art can be suitably used for the non-biodegradable polymer corresponding to the required properties thereof, and examples thereof include, but are not limited to, polymethyl methacrylate (PMMA), polyethyl methacrylate (PEMA), polyvinylpyrrolidone (PVP), polyurethane, polyester, polyolefin, polystyrene, polyvinyl chloride, polyvinyl ether, polyvinylidene fluoride, polyfluoroalkene, nylon and silicone. The skeleton of the non-biodegradable polymer may form a block copolymer with a biodegradable polymer skeleton or may be random polymerized with a monomer unit that forms a biodegradable polymer. In addition, the non-biodegradable polymer may form a copolymer with a plurality of non-degradable polymers in order to obtain a desired property.

One type of structure introduced into a side chain moiety of the polymer of the present invention contains a cationic group for the purpose of imparting antibacterial activity to the polymer. Polymeric cationic peptides are known to be antibacterial substances capable of overcoming bacterial resistance. Cationic peptides such as magainin or cecropin interact with the microbial membrane to cause irreparable damage to the microbial membrane based on electrostatic interaction thereof instead of damaging a specific target protein present in the microorganism. Disruption of the microbial cell membrane ultimately leads to cell death. Several cationic block copolymers have been reported that mimic the amphiphilic structure on the surface of these peptides as well as their antibacterial activity (see, for example, Chem. Eur. J., 2009, 15, 11715-11722, Biomacromolecules, 2009, 10, 1416-1428, Biomacromolecules 2011, 12, 3581-3591, Biomacromolecules, 2012, 13, 1554-1563, and Biomacromolecules, 2012, 13, 1632-1641). Examples thereof include antibacterial polynorbornene and polyacrylate derivatives, poly(arylamide), poly(β-lactam) and pyridinium copolymers. In the present invention, although there are no particular limitations on the cationic group present in the side chain moiety, a primary to quaternary ammonium group is preferable from the viewpoints of ease of introduction into the side chain moiety and control of overall polymer configuration.

In the present invention, a primary to quaternary ammonium group refers to a group that has become a cation as a result of one to four carbon atoms, respectively, bonding to a single nitrogen atom. However, the plurality of carbon atoms are not necessarily required to be different carbon atoms and the case of these carbon atoms being the same carbon atoms is included. For example, a quaternary ammonium group may contain an unsaturated quaternary ammonium core in the manner of a pyridinium group or imidazolium group. A quaternary ammonium group can be present regardless of the pH of the aqueous solution, and is distinguished from protonated lower ammonium groups only under acidic pH conditions. In contrast, primary to tertiary ammonium groups undergo a change in properties according to the pH conditions of the aqueous solution, and primary to tertiary ammonium groups having a positive charge are thought to be in a state of equilibrium with amine groups not having a positive charge under neutral conditions. Thus, these ammonium groups are thought to act as mild cationic groups in the body in comparison with quaternary ammonium groups, and demonstrate effective antimicrobial activity while inhibiting cytotoxicity.

In a more preferable embodiment of the present invention, a polymer is provided that has a primary ammonium group for the aforementioned cationic group in a side chain moiety thereof. Although depending on the application of the resulting polymer, these cationic groups can be present at an arbitrary ratio throughout the entire polymer, and for example, are present at a ratio of about 1 mol % to about 99 mol %, preferably about 5 mol % to about 75 mol %, and even more preferably about 10 mol % to about 50 mol % based on the total amount of the side chain moiety.

A structure expected to demonstrate biocompatibility is introduced into a side chain moiety of the polymer of the present invention along with the aforementioned structure containing a cationic group. A structure capable of being contained by water molecules in a state referred to as “intermediate water” can be used for the structure expected to demonstrate biocompatibility in a polymer in which this structure is incorporated as a side chain moiety of a suitable main chain (see, for example, Tanaka, M. et al., J. Biomat. Sci. Polym. Ed., 2010, 21, 1849-1863). Specific examples of such structures expected to demonstrate biocompatibility include MPC polymers having a side chain containing a phospholipid polar group on a main chain, PEG composed of an ether structure, polymers in the form of PMEA having a side chain mainly composed of an ether structure, and polymer materials having hydrophilic groups such as an ether structure or amide bond in the manner of polyalkoxyalkyl (meth)acrylamide having an ether structure and amide bond therein.

According to prior research, substances that demonstrate biocompatibility have clearly been determined to have the potential to contain “intermediate water” regardless of whether they are biological substances or artificially synthesized substances. The presence of water molecules in this state referred to as intermediate water on the surface of a substance has been experimentally determined to prevent non-specific adsorption of protein present in biological tissue, and as a result thereof, demonstrate biocompatibility. In order for a prescribed substance to contain “intermediate water”, in addition to substances in which the entire substance has a structure suitable for containing “intermediate water” in the manner of PEG, “intermediate water” has been clearly determined to be able to be contained throughout the entire substance by using an alkyl chain and the like for the main chain and providing a structure suitable for containing “intermediate water” for the side chain.

The presence of intermediate water contained in a substance is typically characterized by the unique release and absorption of latent heat observed during the course of heating after supercooling. In other words, in a substance containing intermediate water, during the course of gradually heating to the vicinity of room temperature after having cooled to about −100° C., release of latent heat is observed in the vicinity of −40° C. or absorption of latent heat is observed from −10° C. to the freezing point of water, thereby demonstrating unique release and absorption of latent heat. Various tests have clearly demonstrated that this release and absorption of latent heat is attributable to the presence of regularity or irregularity in the constant ratio of water molecules contained in a substance, and water molecules that behave in this manner are defined as intermediate water. Although intermediate water is presumed to consist of water molecules that are weakly restrained by the specific effects of molecules that compose a substance, since it has also been clearly demonstrated to be contained in biological materials such as phospholipids, it is thought to be involved in the prevention of non-specific adsorption of proteins and the like in biological tissue. The fact that intermediate water can be contained in PMC polymers provided with side chains in the form of phospholipid polar groups contained in the body, as well as in substances such as the aforementioned PEG, PMEA or polyalkoxyalkyl (meth)acrylamides, it is thought to be related to expression of biocompatibility.

In the present invention, although there are no particular limitations on the structure expected to demonstrate biocompatibility that is introduced into a side chain moiety, a preferable example thereof is a group containing at least one ether group from the viewpoints of ease of introduction into the side chain moiety and control of overall polymer configuration.

In the present invention, the structure of the linker moiety that links the aforementioned structure containing a cationic group and the structure expected to demonstrate biocompatibility to the main chain moiety can be suitably determined in consideration of such factors as ease of production within a range that does not impair the properties demonstrated by these side chain structures. Although an ester bond is typically used for the linker and is useful in terms of production of the monomer used, the linker is not limited thereto, but rather a linking form such as an ether bond or amino bond can also be employed.

Preferable examples of the polymer according to the present invention include conventionally known biodegradable polymers having a main chain containing repeating units in which alkylene groups and the like are bound through carbonate bonds, ester bonds, urethane bonds, urea bonds or amide bonds in the same manner as those of aliphatic polyester-based and polyamide-based polymers. Examples of the polymer include those in which side chains containing structures containing a cationic group in the form of primary to quaternary ammonium groups and structures expected to demonstrate biocompatibility in the form of ether structures are introduced by a prescribed bonding mode into carbon atoms contained in an alkylene group and the like.

The fact that polymers containing the aforementioned quaternary ammonium group have antibacterial activity and the fact that polymers having a side chain containing an ether structure demonstrate biocompatibility have been known in the past. In contrast, the polymer of the present invention is based on the finding that antibacterial activity can be imparted while retaining biocompatibility by, for example, using both of these components as side chains and introducing them into a main chain in which alkylene groups are bound by carbonate bonds. In other words, in contrast to hemolysis accompanying destruction of erythrocytes being unable to be reliably inhibited despite being able to obtain bactericidal effects against Escherichia coli and the like in the case of containing only a side chain that demonstrates antibacterial activity, hemolysis and the like were able to be prevented while maintaining bactericidal effects by additionally introducing a structure demonstrating biocompatibility at a suitable ratio.

This type of phenomenon indicates that a portion of the action of quaternary ammonium groups and the like on biological materials in the manner of Escherichia coli or erythrocytes can be alleviated by containing intermediate water through the use of an ether structure and the like. This also indicates that a target substance can be attacked while suppressing hemolysis and other adverse side effects by utilizing the mutual competition between the actions demonstrated by each of these structures.

Although there are no particular limitations on the degree of polymerization of the polymer of the present invention, the average molecular weight of the polymer also changes corresponding to the degree of polymerization and ease of manipulation when using as a material also changes corresponding to molecular weight. In other words, although the polymer according to the present invention is preferably made to have a comparatively small average molecular weight in the case of using as a water-soluble pharmaceutical agent, it preferably is made to have a comparatively large molecular weight to prevent elution in the case of coating onto the surface of various types of base materials. In general, the average molecular weight of the polymer according to the present invention is preferably within the range of 1,000 to 1,000,000, more preferably within the range of 5,000 to 800,000, and most preferably within the range of 8,000 to 500,000. Although there are no particular limitations thereon, the molecular weight distribution of the polymer according to the present invention is preferably within the range of 1.0 to 10, more preferably within the range of 1.0 to 8, and most preferably within the range of 1.05 to 5.0.

The polymer according to the present invention can typically be synthesized by copolymerizing at least two types of monomers consisting of a monomer having a structure containing a cationic group and a monomer having a structure expected to demonstrate biocompatibility. Although the types and usage ratio of monomers used are suitably selected according to the polymer application and required properties, the polymer of the present invention can be obtained by containing at least one type of monomer having a structure provided with the aforementioned properties. A polymerization method known among persons with ordinary skill in the art can be used for the polymerization method corresponding to the monomers used provided the aforementioned plurality of monomers can be copolymerized, and examples thereof include cationic polymerization, anionic polymerization, ring-opening metathesis polymerization and living radical polymerization.

As a specific example of a synthesis method, the polymer according to the present invention can be produced by copolymerizing by ring-opening polymerization at least two types of monomers having a cyclic structure like that shown below preliminarily introduced with a moiety serving as a side chain of a polymer obtained by polymerization.

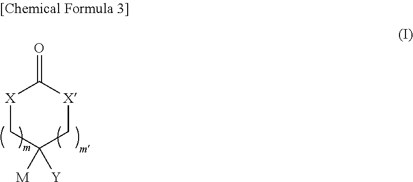

In the aforementioned general formula (I), for example, any of a carbonate bond (O/O), ester bond (CH₂/O), urethane bond (O/N), amide bond (CH₂/N) or urea bond (N/N) is selected for the skeleton moiety contained in the side chain of the polymer following polymerization by selecting CH₂, O or N for the X and X′ adjacent to the carbonyl carbon.

In addition, the length of the alkylene group moiety bound to the skeleton moiety of the main chain is determined by mutually and independently selecting an integer including 0 for m and m′ (although one of these is not zero in the case of a urea bond).

A side chain having Z¹ or Z² in the side chain moiety of the polymer following polymerization can be provided by using at least two types of monomers, in which at least two types of side chain moieties in the form of Z¹ and Z² are separately bound via a linker L, for “Y” in the aforementioned general formula (I). Here, Z¹ represents a side chain moiety having a cationic group or a side chain moiety having a group serving as a precursor of a cationic group, and is preferably selected from among primary to quaternary ammonium groups. On the other hand, Z² represents a group that enables intermediate water to be retained in the body, and preferably includes an ether group. The biocompatible cationic polymer according to the present invention can be produced by mutually polymerizing these at least two types of monomers by ring-opening polymerization at any of the bonds adjacent to the carbonyl carbon. The side chain moiety Z¹ may have a group serving as a precursor of a cationic group instead of a cationic group. For example, Z¹ may contain a functional group capable of forming an ammonium group such as a quaternary ammonium group by reacting with an amine such as a tertiary amine, or may contain a group in which an amine is protected by a protecting group such as a tert-butoxycarbonyl (Boc) group. Cations can be generated from these precursors after obtaining in the form of a polymer by subjecting to further treatment such as de-protection following the polymerization reaction, and a lower ammonium group can be formed by polymerizing while still containing a protecting group added to the ammonium group followed by subjecting to acid hydrolysis following polymerization.

In a preferable embodiment, general formula (I) represents:

a monomer in which X and X′ are both oxygen atoms that form a cyclic carbonate, m and m′ are both 1, and M is a methyl group, and

if a monomer having a prescribed primary to quaternary ammonium group used as Z¹ and a monomer, in which a prescribed ether structure is bound, is used as Z² are mixed at a prescribed molar ratio, a polymer can be obtained by ring-opening polymerization of a cyclic carbonate that has a main chain in which alkylene groups having 3 carbon atoms are bound by carbonate bonds and is respectively provided with the aforementioned primary to quaternary ammonium group and the ether structure as side chains of the central carbon atoms of the alkylene groups. The blending ratio of these two types of monomers in terms of the molar ratio thereof can be 1:99 and 99:1, preferably 10:90 to 90:10 and more preferably 30:70 to 70:30.

In addition, the polymerization method may consist of preliminarily polymerizing the first monomer followed by adding the second polymer and polymerizing to obtain a block copolymer, or may consist of mixing both monomers followed by polymerizing simultaneously to obtain a random copolymer.

In the present invention, among the previously described monomer compounds used, examples of those having Z¹, or in other words, monomers that serve as the moiety responsible for antibacterial activity, include, but are not limited to:

2-(tert-butoxycarbonylamino)ethyl 5-methyl-2-oxo-1,3-dioxane-5-carboxylate, 2-(tert-butoxycarbonylamino)propyl 5-methyl-2-oxo-1,3-dioxane-5-carboxylate, and 2-(benzylamino)ethyl 5-methyl-2-oxo-1,3-dioxane-5-carboxylate, and the monomer used can be suitably selected corresponding to the structure of the target polymer.

In the present invention, among the previously described monomers used, examples of monomers having Z², or in other words, monomers, or monomers serving as precursors thereof, that serve as the moiety responsible for biocompatibility in the polymer include, but are not limited to:

5-methyl-5-(2-methoxyethypoxycarbonyl-1,3-dioxan-2-one, 5-methyl-5-(2-ethoxyethypoxycarbonyl-1,3-dioxan-2-one, 5-methyl-5-(2-tetrahydrofuranylmethyl)oxycarbonyl-1,3-dioxan-2-one, 5-methyl-5-(3-tetrahydrofuranylmethyl)oxycarbonyl-1,3-dioxan-2-one, 5-methyl-5-(3-tetrahydropyranylmethyl)oxycarbonyl-1,3-dioxan-2-one, 5-methyl-[2-(2-methoxyethoxy)ethyl]oxycarbonyl-1,3-dioxan-2-one, 5-methyl-5-(2-epoxyoxyethyl)oxycarbonyl-1,3-dioxan-2-one, 4-methyl-4-(2-methoxyethypoxycarbonyl-1,3-dioxan-2-one, 4-methyl-4-(2-ethoxyethypoxycarbonyl-1,3-dioxan-2-one, 4-methyl-4-(2-tetrahydrofuranylmethyl)oxycarbonyl-1,3-dioxan-2-one, 4-methyl-4-(3-tetrahydrofuranylmethyl)oxycarbonyl-1,3-dioxan-2-one, 4-methyl-4-(3-tetrahhydropyranylmethyl)oxycarbonyl-1,3-dioxan-2-one, 4-methyl-[2-(2-methoxyethoxy)ethyl]oxycarbonyl-1,3-dioxan-2-one, 4-methyl-4-(2-epoxyoxyethyl)oxycarbonyl-1,3-dioxan-2-one, γ-methyl-γ-(2-methoxyethyl)oxycarbonyl-δ-valerolactone, γ-methyl-γ-(2-ethoxyethypoxycarbonyl-δ-valerolactone, γ-methyl-γ-(2-tetrahydrofuranylmethypoxycarbonyl-δ-valerolactone, γ-methyl-γ-(3-tetrahydrofuranylmethyl)oxycarbonyl-δ-valerolactone, and γ-methyl-γ-(3-tetrahydropyranylmethypoxycarbonyl-δ-valerolactone, and the monomer used can be suitably selected corresponding to the structure of the target polymer.

Although the above description has provided an explanation of a method for producing the biocompatible polymer according to the present invention by mixing monomers introduced with a bond such as a carbonate bond expected to demonstrate biocompatibility and a structure containing a prescribed cationic group and group able to retain intermediate water in the body, the present invention is not limited thereto, but rather the biocompatible polymer according to the present invention may also be produced by introducing a prescribed cationic group and group able to retain intermediate water in the body into prescribed carbon atoms serving as the polymer main chain. In the biocompatible polymer composition of the present invention, although it is not always necessary to bond a side chain in the form of a structure containing a cationic group and group able to retain intermediate water in the body to all repeating units of the main chain polymer, a polymer is preferably produced by polymerizing two or more types of monomers introduced with structures containing these groups from the viewpoints of synthetic simplicity and ease of predicting the properties of the polymer.

In addition, in one aspect of the present invention, any of a biodegradable polymer and non-biodegradable polymer can be contained in the main chain moiety. A polymer having such a structure can be obtained by, for example, copolymerizing a biodegradable polymer and a non-biodegradable polymer.

In the compound represented by general formula (I), in the case X and X′ are both —O—, namely in the case of a cyclic carbonate, such a compound can be synthesized using a method known among persons with ordinary skill in the art. For example, as indicated in Scheme 2 below, such a compound can be synthesized by a process starting from a diol derivative and comprising a step (a) consisting of a reaction for introducing a structure containing an ether group, and a step (b) consisting of a reaction for forming a cyclic carbonate through the action of a carbon source such as carbon monoxide in the presence of biphenyl carbonate or catalyst.

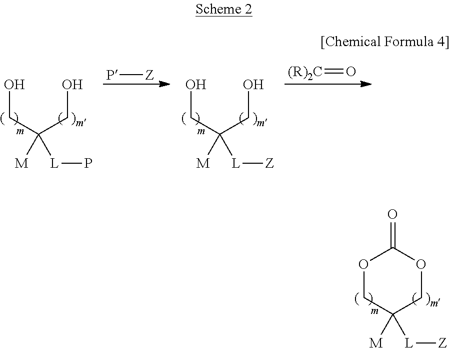

(In the above formula, M, m, m′, L and Z are as previously defined, P and P′ represent leaving groups, and R represents an O-phenyl group, chlorine atom or is not present.)

In still another example, a compound represented by general formula (I), in which the linker moiety L is an ester bond, can be obtained by carrying out step (a), consisting of forming a bis(hydroxy)ester by allowing an alcohol having a structure containing an ether group such as 2-methoxyethanol to act on a carboxylic acid having a diol structure such as 2,2-bis (methylol)propionic acid, followed by step (b), consisting of allowing triphosgene to act thereon.

The step for synthesizing the bis(hydroxy)ester is carried out by, for example, heating in the presence of an ion exchange resin in a solvent depending on the case. In the case of using a solvent, although there are no particular limitations thereon provided the solvent dissolves the raw materials without inhibiting the reaction, the raw material in the form of an alcohol having structural moiety Z can be used as a solvent when the raw material is a liquid and it adequately dissolves the diol. Although the reaction temperature can be within the range of room temperature to the boiling point of the solvent, a temperature within the range of 50° C. to 90° C. is most preferable. Although varying according to the raw material compounds and heating temperature, the reaction time is within the range of 1 hour to 100 hours and preferably within the range of 10 hours to 50 hours.

In the case the linker moiety L is a structure other than an ester structure, the corresponding diol derivative is synthesized by making suitable changes when selecting the raw material compounds, such as by changing the alcohol having the structural moiety Z to an amine in the case L is an amide, or changing the carboxylic acid having a diol structure to a halide in the case L is an ether group (—O—). The reaction conditions used at that time are known among persons with ordinary skill in the art.

The step for forming the cyclic carbonate is carried out by allowing triphosgene to act on the aforementioned resulting diol derivative in a suitable solvent and in the presence of a base. There are no particular limitations on the solvent used, and examples thereof include, but are not limited to, a halogen-based solvent such as dichloromethane or chloroform, an ether-based solvent such as diethyl ether, tetrahydrofuran or 1,4-dioxane, an aromatic solvent such as benzene or toluene, or ethyl acetate and the like. The base is used to generate phosgene in the reaction system by decomposing triphosgene. Examples of base used include, but are not limited to, triethylamine, diisopropylethylamine and pyridine and the like.

In a compound represented by general formula (I), in the case one of X and X′ is —O—, namely in the case of lactone, such a compound can be synthesized using a method known among persons with ordinary skill in the art.

A lactone represented by general formula (I) is synthesized by a method comprising a step (a) a reaction for introducing a structure containing an ether group, and a step (b) a lactonization reaction. The reaction for introducing a structure containing an ether group is as was previously described in the section on carbonate synthesis. The lactonization reaction is carried out using a reaction known among persons with ordinary skill in the art, such as a condensation reaction in the manner of iodolactonization or Staudlinger's ketene cycloaddition reaction, an oxidation reaction using a peracid in the manner of Baeyer-Villiger's oxidation of cyclic ketenes, or oxidation of a preliminarily cyclized lactol. An oxidation method using a peracid is preferable from the high degree of versatility with respect to synthesis of various monomer compounds. For example, a lactone represented by general formula (I) can be synthesized in accordance with Scheme 3 indicated below. The raw material compounds are commercially available or can be obtained according to a synthesis method known among persons with ordinary skill in the art.

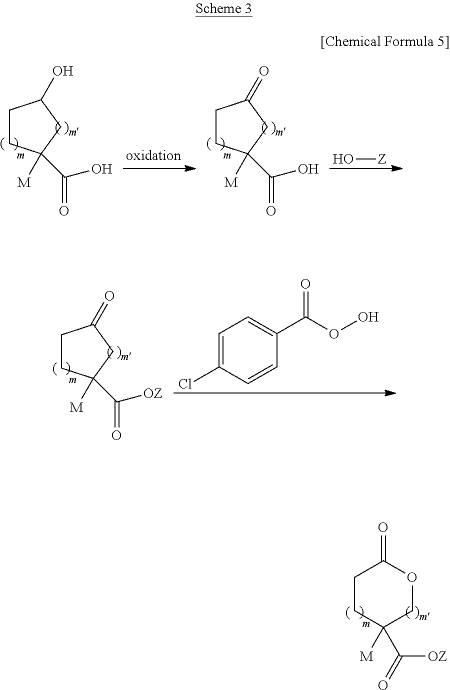

(In the above formula, M, m, m′ and Z are the same as previously defined.)

The polymer produced according to the present invention can be used by adding an additive such as a radical scavenger, peroxide decomposer, antioxidant, ultraviolet absorber, heat stabilizer, plasticizer, flame retardant or antistatic agent as necessary within a range that does not deviate from the gist of the present invention. In addition, it can also be used by mixing with a polymer other than the polymer of the present invention. Such a composition containing the polymer of the present invention is also an object of the present invention.

In the case of using the polymer composition of the present invention as a composition obtained by mixing with other high molecular weight compounds and the like, they can be used at a suitable mixing ratio corresponding to the usage application thereof. By making the ratio of the polymer composition of the present invention to be 90% by weight or more in particular, a composition can be obtained that strongly possesses the characteristics of the present invention. In addition, by making the ratio of the polymer composition of the present invention to be 50% by weight to 70% by weight depending on the usage application, a composition can be obtained that has various properties while still taking advantage of the characteristics of the present invention.

One aspect of the present invention is a medical device obtained by coating with the polymer of the present invention. In addition, the polymer of the present invention can be in the form of a medical device by applying to at least a portion of the surface of a medical device that is used in contact with biological tissue or blood. In other words, in addition to being able to be used as a surface treatment agent applied to the surface of a base material serving as a medical device, it can also be used as a material that composes at least one member of a medical device. Here, a “medical device” includes a device implanted in the body such as an artificial organ and a device such as a catheter that makes temporary contact with biological tissue, and is not limited to that which is manipulated within the body. In addition, a medical device of the present invention is a device used in medical applications which has the polymer of the present invention on at least a portion of the surface thereof. The surface of a medical device as referred to in the present invention refers to, for example, the surface of a material that composes a medical device that contacts blood and the like during use of the medical device, or the surfaces of pores within such a material.

Furthermore, in the present description, the phrase “used in contact with biological tissue or blood” naturally includes, for example, a state of being placed in the body, a state of being used in contact with biological tissue or blood with the biological tissue exposed, and a state of being used in contact with a biological component in the form of blood that has been removed outside the body in a medical material for extracorporeal circulation. In addition, the phase “used in medical applications” includes, for example, the aforementioned “used in contact with biological tissue or blood” and use for which such use is scheduled.

In the present invention, there are no particular limitations on the material or shape of the base material that composes a medical device, and may be, for example, a porous body, fibers, non-woven fabric, particles, film, sheet, tube, hollow fibers or powder. Examples of materials thereof include natural polymers such as cotton or hemp, synthetic polymers such as nylon, polyester, polyacrylonitrile, polyolefin, halogenated polyolefins, polyurethane, polyamide, polycarbonate, polysulfone, polyether sulfone, poly(meth)acrylate, ethylene-vinyl alcohol copolymer, butadiene-acrylonitrile copolymer, and mixtures thereof. In addition, other examples include metals, ceramics and composite materials thereof, the base material may be composed of a plurality of base materials, and the polymer composition according to the present invention is preferably provided on at least a portion of the surface, and preferably over nearly the entire surface thereof, that makes contact with blood.

In a preferable embodiment of the present invention, the polymer can be used as a material serving as the entirety of a medical device used in contact with biological tissue or blood or as a material serving as the surface thereof, and at least a portion of the surface in contact with blood, and preferably nearly all of the surface in contact with blood, of a medical device such as an artificial organ or therapeutic device implanted in the body, an extracorporeal circulation type of artificial organ, surgical suture or catheter (in the manner of a circulatory catheter such as an angiographic catheter, guide wire or PTCA catheter, digestive tract catheter such as a gastric tube catheter, gastrointestinal catheter or esophageal catheter, or urological catheter such as a tube, urethral catheter or ureteral catheter) is preferably composed of the polymer according to the present invention. In addition, the polymer can be particularly preferably used in a medical device that uses the polymer according to the present invention in the form of a biodegradable polymer and is implanted in the body during treatment.

The polymer composition of the present invention may also be used in a hemostatic agent, tissue adhesive material, repair material for tissue regeneration, carrier of a drug sustained-release system, hybrid artificial organ such as an artificial pancreas or artificial liver, artificial blood vessel, embolic material or matrix material for providing a scaffold for cellular engineering.

Examples of methods used to retain a composition containing the polymer of the present invention on the surface of a medical device and the like include known methods such as a coating method, graft polymerization using radiation, electron beam or ultraviolet rays, and a method for introducing the polymer of the present invention by utilizing a chemical reaction with a functional group of the base material. Among these, a coating method is particularly preferable in practical terms since the production operation is easy. Moreover, although examples of coating methods include coating, spraying and dipping, any of these can be applied without any particular limitations. The film thickness thereof is preferably 0.1 μm to 1 mm. For example, coating treatment by coating the composition containing the polymer of the present invention can be carried out by a simple procedure such as by immersing a member to be coated in a coating solution obtained by dissolving the composition containing the polymer of the present invention in a suitable solvent followed by adequately removing the solvent and drying. In addition, in order to more securely immobilize the polymer of the present invention on a member to be coated, adhesion with the polymer of the present invention can be further enhanced by applying heat after coating. In addition, the polymer may also be immobilized by crosslinking the surface. Introducing a crosslinked polymer as a comonomer component may be employed for the crosslinking method. In addition, the polymer may also be crosslinked by irradiation of an electron beam, gamma rays or light.

In another embodiment of the present invention, an antibacterial agent is provided that contains the polymer of the present invention in which the main chain of the polymer is biodegradable. An antibacterial agent containing the polymer of the present invention can be used, for example, in a liquid state such as an injection solution as an alternative to antibiotics. Since the polymer of the present invention not only demonstrates antibacterial activity based on the presence of a cationic group, but is also imparted with biocompatibility while also being made to have biodegradability, it can be administered into the body in the form of a safe antibacterial agent. Moreover, since polymer composition and properties can be suitably modified according to the selection of monomers, a polymer can be designed for which the amount of time the polymer remains in the body and sustains its antibacterial action has been suitably adjusted corresponding to the application. The present invention is based on the finding that a polymer capable of demonstrating antibacterial activity by having a broad antibacterial spectrum can be designed corresponding to the required properties while retaining biocompatibility.

### EXAMPLES

The following provides a detailed explanation of the polymer of the present invention by indicating examples of synthesis methods thereof and experimental methods used to examine antibacterial activity and hemolytic activity along with the results thereof. The present invention is not limited to these examples.

### Example 1

### Synthesis of 2,2-bis-(hydroxymethyl)-propionic acid benzyl ester (2)

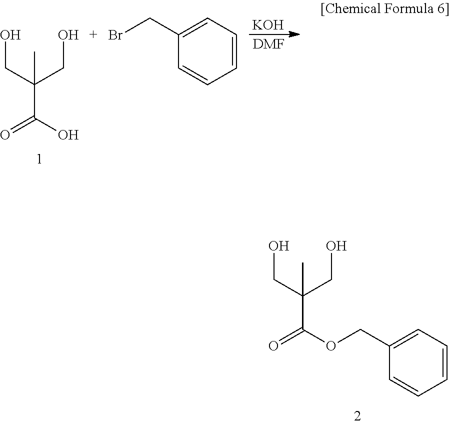

2,2-bis(hydroxymethyl)propionic acid (1) (22.5 g, 0.168 mol), potassium hydroxide (11.0 g, 0.165 mol) and N,N-dimethylformamide (DMF, 125 mL) were added to a 500 mL three-necked flask, which was equipped with a reflux condenser and injected with nitrogen following degassing, followed by stirring for 1 hour at 100° C. After confirming that the solution became clear, benzyl bromide (23.96 mL, 0.202 mol) was added followed by stirring for 16 hours at 100° C. Subsequently, the reaction solution was cooled to room temperature and the resulting precipitate was removed by suction filtration. After concentrating the resulting filtrate with a rotary evaporator, the concentrate was dissolved in ethyl acetate (150 mL) and hexane (150 mL) and washed twice with a separatory funnel using ion exchange water (150 mL). The organic layer was then dried with magnesium sulfate and concentrated with a rotary evaporator. The resulting solid was recrystallized with toluene to obtain 2,2-bis-(hydroxymethyl)-propionic acid benzyl ester in the form of a white solid (amount recovered: 23.3 g, yield: 62.0%). ¹H-NMR (400 MHz, CDCl₃): δ7.37 (m, 5H, ArH), 5.23 (s, 2H, ArCH₂), 3.965 (d, J=12 Hz, 2H, CHₐCH_{b}), 3.75 (d, J=8 Hz, 2H, CHₐCH_{b}), 1.09 (s, 3H, CCH₃).

### Example 2

### Synthesis of 5-methyl-5-benzyloxycarbonyl-1,3-dioxan-2-one (3)

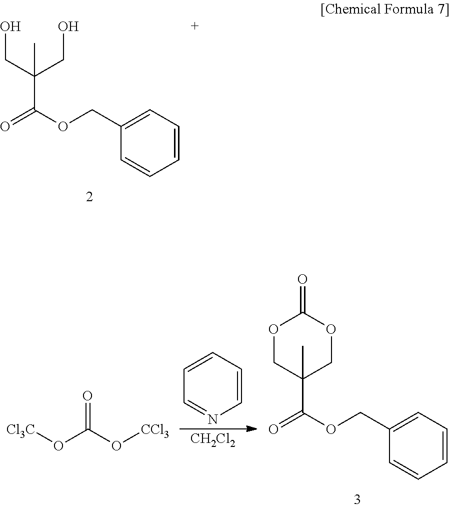

Compound (2) (13.0 g, 58.0 mol) was added to a 500 mL three-necked flask equipped with a 100 mL dropping funnel followed by replacing the air inside the flask with nitrogen after degassing. Next, dried methylene chloride (175 mL) and pyridine (28.0 mL, 348 mmol) were added. The reaction system was cooled to −75° C. using a dry ice/2-isopropanol cooling bath followed by gradually adding a preliminarily prepared dried DCM solution (75.0 mL) of triphosgene (8.61 g, 29.0 mmol) with the dropping funnel. Following completion of dropping, the reaction solution was stirred for 1 hour while cooling to −75° C. followed by stirring for 2 hours at room temperature. Following completion of the reaction, a saturated solution of ammonium chloride (90.0 mL) was added followed by stirring for 30 minutes and washing the organic phase three times with 1 N hydrochloric acid solution (120 mL) and one time each with saturated solution of sodium bicarbonate (120 mL) and saturated solution of sodium chloride (120 mL) using a separatory funnel. The resulting organic layer was dried with magnesium sulfate followed by concentrating under reduced pressure with a rotary evaporator and further subjecting to vacuum drying at room temperature to obtain 5-methyl-5-benzyloxycarbonyl-1,3-dioxan-2-one in the form of a white solid (amount recovered: 13.7 g, yield: 94.4%). ¹H-NMR (400 MHz, CDCl₃): δ7.35 (m, 5H, ArH), 5.21 (s, 2H, ArCH₂), 4.705 (d, J=12 Hz, 2H, CHₐCH_{b}), 4.19 (d, J=12 Hz, 2H, CHₐCH_{b}), 1.33 (s, 3H, CCH₃).

### Example 3

### Synthesis of 5-methyl-2-oxo-1,3-dioxane-5-carboxylic Acid (4)

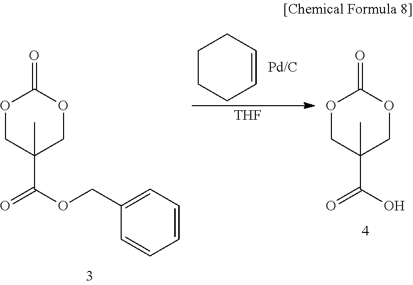

Compound (3) (8.0 g, 32.0 mmol) was dissolved in tetrahydrofuran (THF, 160 mL) in a 500 mL three-necked flask equipped with a reflux condenser followed by adding palladium-carbon (2.0 g, 25% w/w), injected with nitrogen following degassing completely. Then cyclohexene (32.5 mL, 320 mmol) was added and stirring for 24 hours at 60° C. Following completion of the reaction, the solution was cooled to room temperature and hydrogen gas was removed by a degassing procedure. Insoluble matters were filtered out with a glass filter containing diatomaceous earth wetted with THF and the filtrate was concentrated under reduced pressure with a rotary evaporator followed by further vacuum drying at room temperature. The resulting solid was washed with methylene chloride and the remaining residue was isolated by suction filtration to obtain 5-methyl-2-oxo-1,3-dioxane-5-carboxylic acid in the form of a white solid (amount recovered: 4.66 g, yield: 90.9%). ¹H-NMR (400 MHz, acetone-d₆): δ4.665 (d, J=12 Hz, 2H, CHₐCH_{b}), 4.355 (d, J=12 Hz, 2H, CHₐCH_{b}), 1.32 (s, 3H, CCH₃).

### Example 4

### Synthesis of tert-butyl N-(2-hydroxyethyl)carbamate (5)

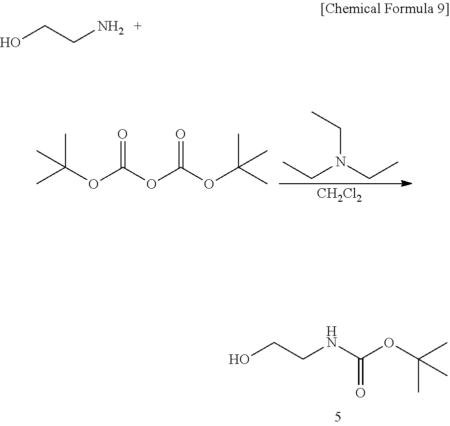

Ethanolamine (3.01 mL, 50.0 mmol), triethylamine preliminarily purified by distillation (7.67 mL, 550 mmol) and dried methylene chloride (35 mL) were added to a 500 mL three-necked flask, which was equipped with a 50 mL dropping funnel and injected with nitrogen following degassing. Next, a dried methylene chloride solution (25 mL) of preliminarily purified di-tert-butyl dicarboxylic acid (10.9 g, 50.0 mmol) was gradually added with the dropping funnel followed by stirring for 18 hours at room temperature. Following completion of the reaction, the solution was concentrated under reduced pressure with a rotary evaporator, and the resulting residue was dissolved in diethyl ether (85 mL) and washed twice with saturated solution of sodium bicarbonate (85 mL) and twice with saturated solution of sodium chloride (60 mL) using a reparatory funnel. Moreover, the aqueous layer was extracted twice with chloroform (150 mL) and combined with the previous organic layer. Subsequently, the organic layer was dried with magnesium sulfate and concentrated under reduced pressure with a rotary evaporator. The concentrate was then adequately vacuum-dried at room temperature to obtain tert-butyl N-(2-hydroxyethyl)carbamate (5) in the form of a colorless, viscous liquid (amount recovered: 7.56 g, yield: 93.9%). ¹H-NMR (500 MHz, acetone-d₆): δ5.90 (br, 1H, NH), 3.555 (q, J=7.5 Hz, 2H, OCH₂), 3.165 (q, J-7.5 Hz, 2H, CH₂NH), 1.40 (s, 9H, C(CH₃)₃).

### Example 5

### Synthesis of 2-(tert-butoxycarbonylamino)ethyl 5-methyl-2-oxo-1,3-dioxane-5-carboxylate (6)

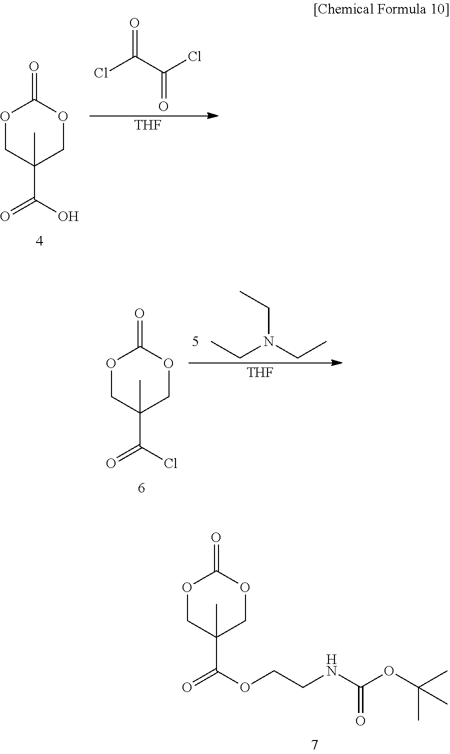

Compound (4)(2.4 g, 15.0 mmol) was added to a 200 mL three-necked flask equipped with a 50 mL dropping funnel followed by degassing the flask, injecting with nitrogen and adding dried THF (75 mL) and several drops of DMF. Next, a preliminarily prepared dried THF solution (30 mL) of oxalyl chloride (1.5 mL, 16.5 mmol) was gradually added with the dropping funnel followed by stirring for 1 hour at room temperature. Following completion of the reaction, after removing acidic gas by bubbling with nitrogen for 30 minutes, the reaction solution was concentrated under reduced pressure with a rotary evaporator. After confirming that the reaction had been completed by ¹H-NMR, the concentrate was again dissolved in dried THF (35 mL) and added to a 200 mL three-necked flask equipped with a 50 mL dropping funnel that had been injected with nitrogen after degassing. After a dried THF solution (25 mL) of Compound (5) that preliminarily dried by adding calcium hydride and stirring overnight and filtered with a syringe filter (0.45 μm), (2.3 g, 14.3 mmol) and triethylamine purified by distillation (2.20 mL, 15.8 mmol) were added to the dropping funnel and gently dropped in. After stirring for 3 hours at room temperature, the precipitate was filtered out and the filtrate was concentrated under reduced pressure with a rotary evaporator. After subsequently adding ethyl acetate (75 mL) to the residue, insoluble matters were removed by suction filtration, the filtrate was washed with 1 N hydrochloric acid solution (75 mL), saturated solution of sodium chloride (75 mL) and ion exchange water (75 mL) with a separatory funnel. After drying the resulting organic layer with magnesium sulfate, the organic layer was concentrated under reduced pressure with a rotary evaporator and vacuum-dried at room temperature. Subsequently, column chromatography was carried out using ethyl acetate for the eluent followed by recrystallizing with a mixture of ethyl acetate and hexane to obtain 2-(tert-butoxycarbonylamino) ethyl 5-methyl-2-oxo-1,3-dioxane-5-carboxylate (7) in the form of a white solid (amount recovered: 1.6 g, yield: 41.9%). ¹H-NMR (400 MHz, acetone-d₆): δ6.21 (br, 1H, NH), 4.69 (d, J=8 Hz, 2H, CHₐCH_{b}), 4.36 (d, J=8 Hz, 2H, CHₐCH_{b}), 4.225 (t, J=6 Hz, 2H, OCH₂CH₂), 3.385 (q, 2H, CH₂CH₂NH), 1.40 (s, 9H, C(CH₃)₃), 1.33 (s, 3H, CCH₃).

### Example 6

### Polymer Synthesis (Homopolymerization of Compound (7))

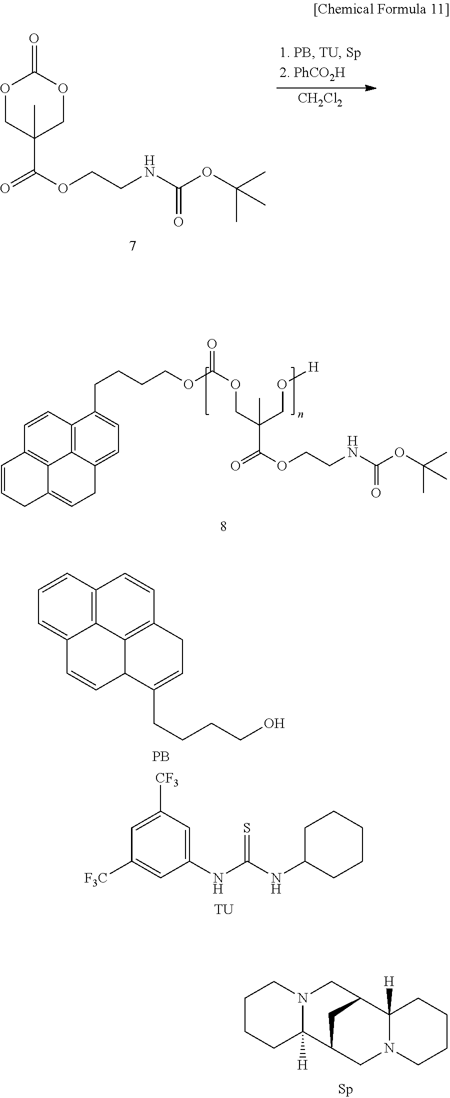

Compound (7) (0.303 g, 1.00 mmol), 1-pyrenebutanol (PB, 5.48 mg, 0.02 mmol), N-bis(3,5-trifluoromethyl)phenyl-N-cyclohexylthiourea (TU, 37.0 mg, 0.10 mmol) and spartine (Sp, 11.7 mg, 0.05 mmol) were dissolved in dried methylene chloride (2 mL) in a glove box in the presence of a nitrogen atmosphere followed by stirring at room temperature. 47.5 hours later, after confirming consumption of the majority of the monomer (83.7%) by ¹H-NMR, benzoic acid was added to terminate polymerization. The reaction solution was subsequently removed from the glove box and re-precipitated in a mixture of hexane and toluene (4/1) followed by isolating the polymer by centrifugal separation. The polymer was subsequently adequately dried in a vacuum to obtain PCBAE (8) in the form of a colorless viscous substance (amount recovered: 0.19 g, yield: 62.1%). GPC (THF): Mn=4,300, Mw/Mn=1.18. ¹H-NMR (500 MHz, CDCl₃): δ8.32-7.78 (m, 9H, pyrene), 5.06 (br, 16H, NH), 4.39-4.25 (m, 76H, OCOOCH₂), 4.24-4.16 (m, 45H, COOCH₂), 3.40-3.31 (m, 34H, CH₂NH), 1.45 (s, 203H, C(CH₃)₃), 1.28 (s, 53H, CCH₃). Average degree of polymerization: n=20.

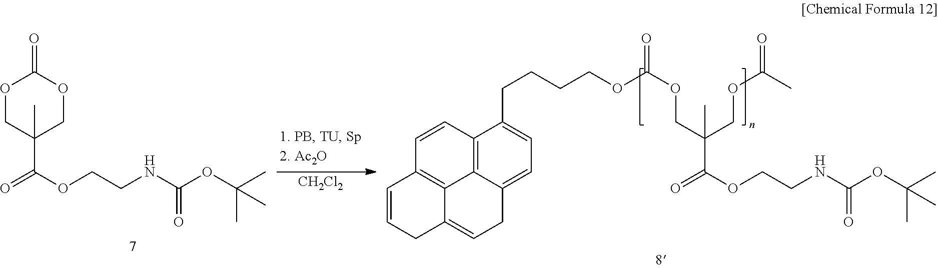

Compound 7 (0.303 g, 1.00 mmol), PB (2.74 g, 0.01 mmol), TU (18.5 mg, 0.05 mmol) and Sp (5.86 mg, 0.025 mmol) were dissolved in dried methylene chloride (1 mL) in a glove box in the presence of a nitrogen atmosphere followed by stirring at room temperature. 22 hours later, after confirming that the majority (85.1%) of the monomer had been consumed by ¹H-NMR, acetic anhydride (Ac₂O) was added to terminate polymerization and simultaneously acetylate the polymer ends. After stirring the polymer solution for 1 hour, the solution was removed from the glove box and re-precipitated in a mixture of hexane and toluene (4/1) followed by isolating the polymer by centrifugal separation. The polymer was subsequently adequately dried in a vacuum to obtain PCBAE (8′) in the form of a colorless viscous substance (amount recovered: 0.222 g, yield: 73.0%). GPC (THF): Mn=9,600, Mw/Mn=1.27. ¹H-NMR (400 MHz, DMSO-d₆): δ8.38-7.66 (m, 9H, pyrene), 6.89 (br, 21H, NH), 4.23 (m, 84H, CH₂OCOO), 4.01 (m, 50H, OCH₂CH₂), 3.16 (m, 50H, CH₂CH₂NH), 1.99 (OCOCH₃ end group), 1.36 (s, 223H, C(CH₃)₃), 1.16 (s, 64H, CH₃). Average degree of polymerization: n=25.

### Example 7

### Copolymerization with MTC-ME (9)

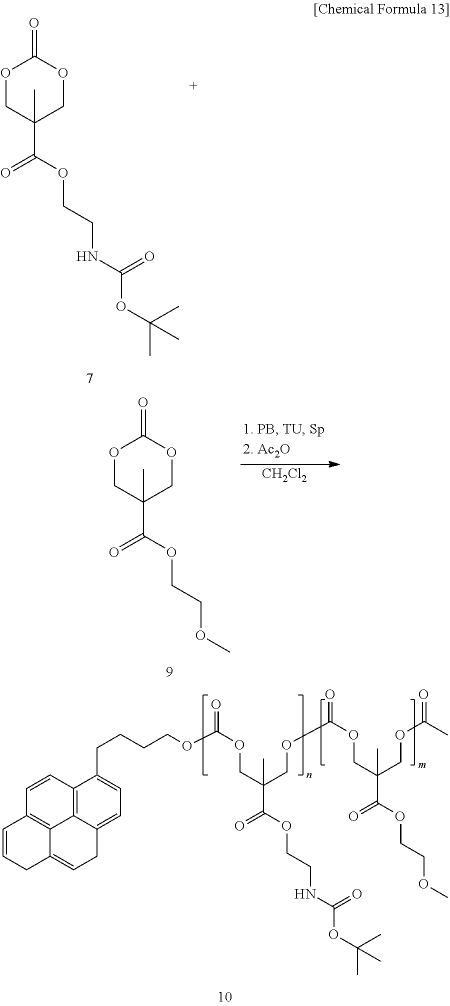

### ((7):(9)=1:1)

MTC-ME (9) was synthesized according to a known method. As a result of introducing MTC-ME, a side chain having a (—CH₂CH₂—O—CH₃) structure was introduced into the polymer. The structure of this side chain is the same as the structure of the side chain moiety of poly(2-methoxyethyl)acrylate (PMEA), which is known to be a side chain that is capable of containing intermediate water and have biocompatibility. Compound (7) (0.303 g, 1.00 mmol), Compound (9) (0.218 g, 1.00 mol), PB (2.74 mg, 0.01 mmol), TU (37.0 mg, 0.10 mmol) and Sp (11.7 mg, 0.05 mmol) were dissolved in dried methylene chloride (2 mL) in a glove box in the presence of a nitrogen atmosphere followed by stirring at room temperature. 72 hours later, after confirming monomer consumption (91.8%) by ¹H-NMR, acetic anhydride was added to terminate polymerization and acetylate the polymer ends. One hour after adding acetic anhydride, the reaction solution was removed from the glove box and re-precipitated in a mixture of hexane and toluene (4/1) followed by isolation of the polymer by centrifugal separation. Subsequently, the polymer was adequately dried in a vacuum to obtain PC(BAE-ME) (10a) in the form of a colorless viscous substance (amount recovered: 0.42 g, yield: 80.1%). GPC (DMF): Mn=10,500, Mw/Mn=1.30. ¹H-NMR (400 MHz, DMSO-d₆): δ8.42-7.61(m, 9H, pyrene), 6.87 (br, 31H, NH), 4.30-4.10 (m, 334H, CH₂OCOO and COOCH₂CH₂), 4.02 (m, 72H, OCH₂CH₂N), 3.49 (m, 75H, COOCH₂CH₂), 3.23 (s, 85H, OCOCH₃), 3.15 (m, 69H, oCH₂CH₂N), 2.00 (s, 3H, OCOCH₃ end group), 1.36 (s, 324H, C(CH₃)₃), 1.17 (s, 194H, CH₃). Average degree of polymerization: n=35, m=38.

### Example 8

### ((7):(9)=1:3)

A copolymer with MTC-ME (9) was synthesized using the same procedure as Example 7. However, Compound (7) (0.152 g, 0.50 mmol) and Compound (9) (0.327 g, 1.50 mmol) were used. 76 hours later, after confirming monomer consumption (86%) by ¹H-NMR, acetic anhydride was added to terminate polymerization and acetylate the polymer ends. One hour after adding acetic anhydride, the reaction solution was removed from the glove box and re-precipitated in a mixture of hexane and toluene (4/1) followed by isolation of the polymer by centrifugal separation. Subsequently, the polymer was adequately dried in a vacuum to obtain PC(BAE-ME) (10b) in the form of a colorless viscous substance (amount recovered: 0.308 g, yield: 64%). GPC (DMF): Mn=8,100, Mw/Mn=1.32. ¹H-NMR (500 MHz, CDCl₃): Average degree of polymerization: n=10, m=28.

### Example 9

### ((7):(9)=1:9)

A copolymer with MTC-ME (9) was synthesized using the same procedure as Example 7. However, Compound (7) (0.061 g, 0.20 mmol) and Compound (9) (0.393 g, 1.80 mmol) were used. 76 hours later, after confirming monomer consumption (87%) by ¹H-NMR, acetic anhydride was added to terminate polymerization and acetylate the polymer ends. One hour after adding acetic anhydride, the reaction solution was removed from the glove box and re-precipitated in a mixture of hexane and toluene (4/1) followed by isolation of the polymer by centrifugal separation. Subsequently, the polymer was adequately dried in a vacuum to obtain PC(BAE-ME) (10c) in the form of a colorless viscous substance (amount recovered: 0.261 g, yield: 57%). GPC (DMF): Mn=8,300, Mw/Mn=1.34. ¹H-NMR (500 MHz, CDCl₃): Average degree of polymerization: n=3, m=25.

### Example 10

### ((7):(9)=5:95)

A copolymer with MTC-ME (9) was synthesized using the same procedure as Example 7. However, Compound (7) (0.030 g, 0.10 mmol) and Compound (9) (0.415 g, 1.90 mmol) were used. 76 hours later, after confirming monomer consumption (88%) by ¹H-NMR, acetic anhydride was added to terminate polymerization and acetylate the polymer ends. One hour after adding acetic anhydride, the reaction solution was removed from the glove box and re-precipitated in a mixture of hexane and toluene (4/1) followed by isolation of the polymer by centrifugal separation. Subsequently, the polymer was adequately dried in a vacuum to obtain PC(BAE-ME) (10d) in the form of a colorless viscous substance (amount recovered: 0.275 g, yield: 61%). GPC (DMF): Mn=9,000, Mw/Mn=1.33. ¹H-NMR (500 MHz, CDCl₃): Average degree of polymerization: n=2, m=26.

### Example 11

### De-Protection of Boc Group

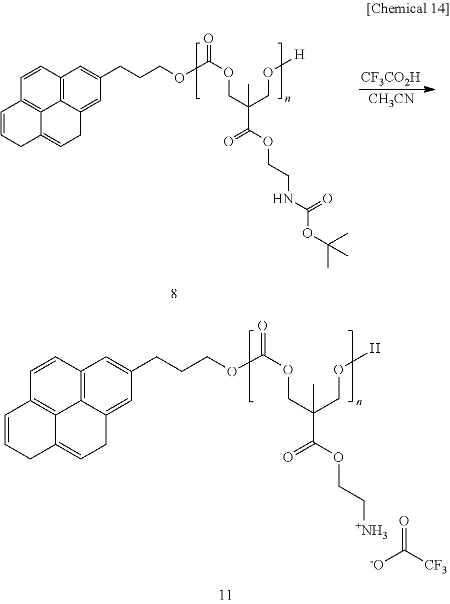

Compound (8) (0.19 g, Boc group: 0.63 mmol) was dissolved in 3.0 mL of acetonitrile and added to a 10 mL Schlenk tube for which the air inside had been replaced with nitrogen after degassing. The reaction system was cooled to −5° C. in cooling bath with an ice water/salt and trifluoroacetic acid (0.63 mL, 8.23 mmol) was gently dropped in under these low-temperature conditions followed by stirring for 30 minutes at −5° C. and subsequently stirring for 6 hours at room temperature. The polymer was subsequently re-precipitated in diethyl ether (30 mL) and the precipitate was recovered by centrifugal separation. The residue was then vacuum-dried at room temperature to obtain PC2PA (11) in the form of a colorless viscous substance (amount recovered: 86.6 mg, yield: 67.6%). GPC (DMF): Mn=1,900, Mw/Mn=3.76. ¹H-NMR (500 MHz, DMSO): δ8.23-7.89 (m, NH₃), 4.34-4.10 (m, 155H, CH₂OCOO and COOCH₂CH₂N), 3.11 (m, CH₂NH₃), 1.25-1.05 (m, CH₃). Zeta potential: +58.9 mV, Dh: 312.2 nm, PDI: 0.597.

### Example 12

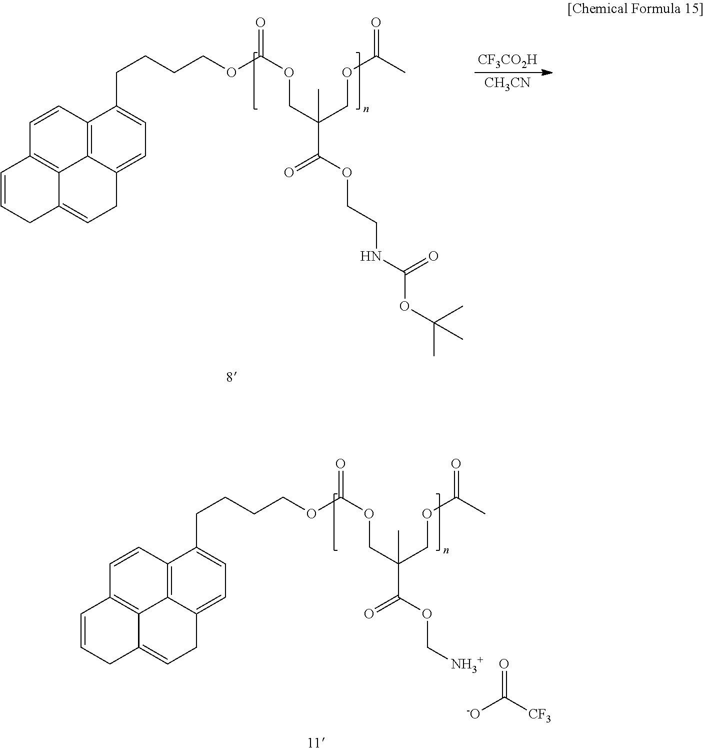

Compound (8′) (0.17 g, Boc group: 0.56 mmol) was dissolved in 3.0 mL of acetonitrile and added to a 10 mL Schlenk tube for which the air inside had been replaced with nitrogen after degassing. The reaction system was cooled to −5° C. in cooling bath with an ice water/salt and trifluoroacetic acid (0.44 mL, 5.64 mmol) was gently dropped in under these low-temperature conditions followed by stirring for 10 minutes at −5° C. and subsequently stirring for 6 hours at room temperature. The polymer was subsequently re-precipitated in a mixture of diethyl ether and hexane (1/1, 30 mL) and the precipitate was recovered by decantation and centrifugal separation. The residue was then vacuum-dried at room temperature to obtain PC2PA (11′) in the form of a colorless viscous substance (amount recovered: 124 mg, yield: 69%). GPC (DMF): Mn=1,500, Mw/Mn=7.37. ¹H-NMR (500 MHz, DMSO): δ8.14 (br, 72H, NH₃), 4.40-4.11 (m, 155H, CH₂OCOO and COOCH₂CH₂), 3.11 (m, 56H, CH₂NH₃), 2.00 (s, 3H, OCOCH₃ end group), 1.20 (m, 80H, CH₃). Average degree of polymerization: n=25.

### Example 13

### ((7):(9)=1:1)

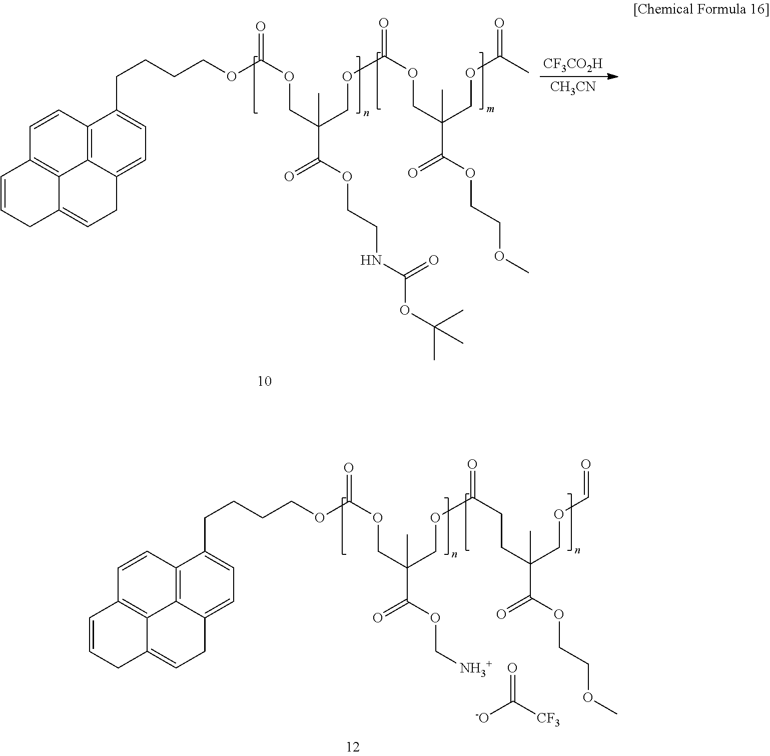

Compound (10a) (0.37 g, Boc group: 0.35 mmol) was dissolved in 6.0 mL of acetonitrile and added to a 10 mL Schlenk tube for which the air inside had been replaced with nitrogen after degassing. The reaction system was cooled to −5° C. in cooling bath with an ice water/salt and trifluoroacetic acid (0.26 mL, 3.52 mmol) was gently dropped in under these low-temperature conditions followed by stirring for 15 minutes at −5° C. and subsequently stirring for 24 hours at room temperature. The polymer was subsequently re-precipitated in a mixture of diethyl ether and hexane (1/1, 60 mL) and the precipitate was recovered by decantation and centrifugal separation. The residue was then vacuum-dried at room temperature to obtain PC(2PA-ME) (12) in the form of a colorless viscous substance (amount recovered: 295 mg, yield: 88.0%). GPC (DMF): Mn=5,200, Mw/Mn=1.38. ¹H-NMR (400 MHz, DMSO): δ8.04 (br, 125H, NH), 4.24-4.10 (m, 524H, CH₂OCOO and COOCH₂CH₂), 3.23 (s, 132H, OCH₃), 3.11 (m, 97H, CH₂NH₃), 2.00 (s, 3H, OCOCH₃ end group), 1.25-1.14 (m, 259H, CH₃). Average degree of polymerization: n=48, m=43. Zeta potential: +17.6 mV, Dh: 337.2 nm, PDI: 0.434.

### Example 14

### Synthesis of PDEAEMA (13)

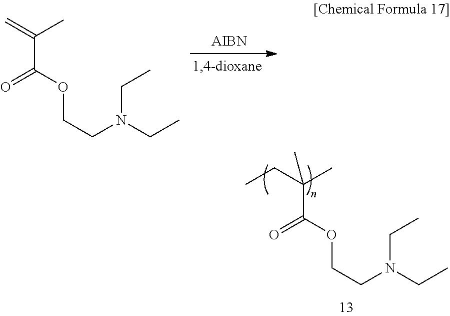

Diethylaminoethyl methacrylate (15.0 g, 81.0 mmol) and azobisisobutyronitrile (AIBN, 1.21 g, 7.36 mmol) were added to a 100 mL three-necked flask, which was equipped with a reflux condenser and injected with nitrogen following degassing, followed by dissolving in THF (30 mL) and stirring at 60° C. After discontinuing stirring 20 hours later and returning to normal temperature, the polymer was re-precipitated in ultrapure water (1 L) and the resulting precipitate was dissolved in THF and again re-precipitated in ultrapure water. This purification procedure was carried out three times. Subsequently, the ultrapure water was removed by decantation followed by vacuum-drying at room temperature to obtain PDEAEMA (amount recovered: 13.58 g, yield: 90.5%). ¹H-NMR (400 MHz, MeOH-d₄): δ4.06 (br, COOCH₂CH₂N), 2.90-2.73 (m, COOCH₂CH₂N), 2.72-2.50 (m, NH(CH₂CH₃)₂), 2.17-1.76 (m, COOCH₂CH₂N), 1.23-0.80 (m, CH₃). GPC(DMF): Mn=17,600, Mn/Mw=1.87.

### Example 15

### Synthesis of PDEAEMA-PMEA Random Copolymer (14)

Poly(2-methoxyethyl acrylate) (PMEA) is a substance that is capable of containing intermediate water and have biocompatibility.

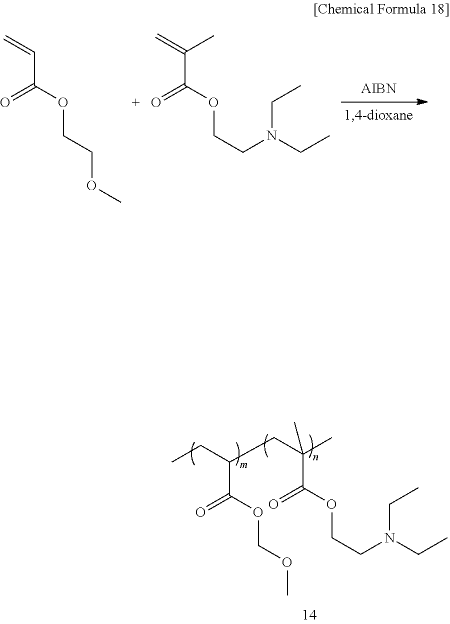

Methoxyethyl acrylate (10.16 g, 78.1 mmol), diethylaminoethyl methacrylate (4.826 g, 26.0 mmol), and azobisisobutyronitrile (AIBN, 15 mg, 9.13×10⁻² mmol) were added to a 300 mL three-necked flask, which was equipped with a reflux condenser and injected with nitrogen following degassing, followed by dissolving in 1,4-dioxane (60 g, 62 mL) and stirring at 75° C. After discontinuing stirring 24 hours later and returning to normal temperature, the polymer was re-precipitated in hexane (1 L) and the resulting precipitate was dissolved in THF and again re-precipitated in hexane. Subsequently, the hexane was removed by decantation and the precipitate was dissolved in THF, concentrated under reduced pressure with a rotary evaporator and then vacuum-dried at room temperature. After drying, the substance was purified with ultrapure water to obtain P(MEA-DEAEMA) (amount recovered: 3.95 g, yield: 26.1%). ¹H-NMR (400 MHz, MeOH-d₄): δ4.34-3.96 (m, COOCH₂CH₂), 3.61 (s, COOCH₂CH₂O), 3.38 (s, OCH₃), 2.91-2.56 (m, NCH₂), 2.54-1.52 (m, CH₂), 1.26-0.89 (m, CH₃ and CH). GPC(DMF): Mn=11,000, Mn/Mw=1.67.

### Example 16

### Tertiary Ammonium Chloride

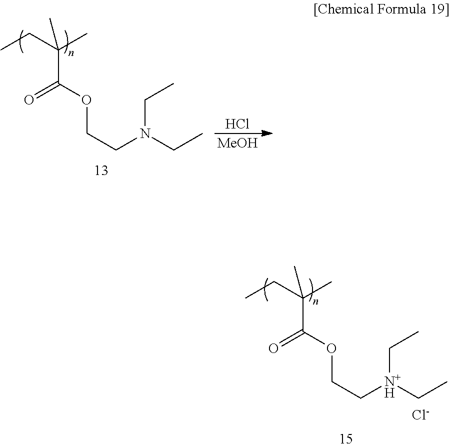

Compound (13) (500 mg, N 5.40 mmol) was dissolved in 2.7 mL of methanol in a 10 mL test tube having a side arm for which the air inside had been replaced with nitrogen after degassing followed by cooling in an ice bath (containing salt and ice water). Concentrated hydrochloric acid (10 N, 1.35 mL, 13.5 mmol) was dropped in over the course of 3 minutes using a Pasteur pipette followed by stirring for 2.5 hours at room temperature. After confirming completion of the reaction by ¹H-NMR, the polymer was re-precipitated in diethyl ether followed by recovery of the precipitate by decantation and vacuum-drying at room temperature to obtain Compound (15) in the form of a clear, viscous substance (amount recovered: 284.3 g, yield: 51%). ¹H-NMR (500 MHz, MeOH): δ4.5 (br, COOCH₂CH₂N), 3.58 (s, COOCH₂CH₂N), 3.36 (m, NH(CH₂CH₃)₂), 2.36-1.63 (m, CH₂), 1.44 (s, CH₂CH₃), 1.27-0.91 (m, CH₃). Zeta potential: +33.5 mV. Dh: 149.5 nm, PDI: 0.908.

### Example 17

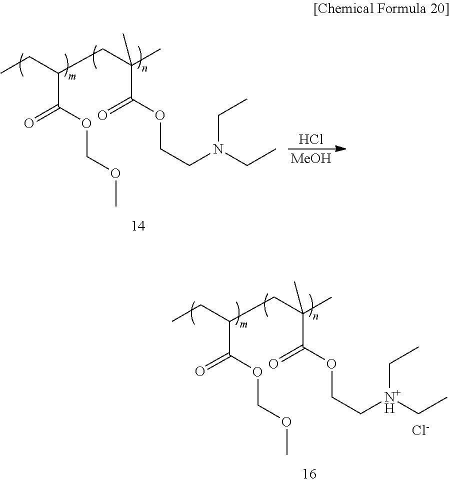

Compound (14) (500 mg, N 1.11 mmol) was dissolved in 6.74 mL of methanol in a 10 mL test tube having a side arm for which the air inside had been replaced with nitrogen after degassing followed by cooling in an ice bath (containing salt and ice water). Concentrated hydrochloric acid (10 N, 0.56 mL, 5.55 mmol) was dropped in over the course of 3 minutes using a Pasteur pipette followed by stirring for 1 hour at room temperature. After confirming completion of the reaction by ¹H-NMR, the polymer was re-precipitated in diethyl ether followed by recovery of the precipitate by decantation and vacuum-drying at room temperature to obtain Compound (16) in the form of a clear, viscous substance (amount recovered: 496.2 g, yield: 92%). ¹H-NMR (500 MHz, MeOH): δ4.50-4.00 (m, COOCH₂CH₂), 3.61 (s, COOCH₂CH₂O), 3.47 (br, COOCH₂CH₂N), 3.37 (s, OCH₃), 2.60-1.44 (m, CH₂), 1.38 (s, CH₃), 1.26-0.98 (m, CH). Zeta potential: +27.7 mV. Dh: 307.4 nm, PDI: 0.548.

### Example 18

### Antibacterial Activity Test

Agar was added to LB medium (tryptone: 1 w/v%, yeast extract: 0.5 w/v % and sodium chloride: 0.5 w/v % dissolved in sterile ultrapure water) to a concentration of 1.5 w/v % to prepare an LB plate. Escherichia coli (Takara Bio, Inc., E. coli DH5α Competent Cells, Product Code: 9057) was spread onto the LB plate and cultured overnight at 37° C. A single colony on the plate was picked off and inoculated into 80 mL of LB medium followed by shake-culturing overnight under conditions of 37° C. and 230 rpm. The bacterial cell concentration was then adjusted to an OD₆₀₀ value of 0.2 based on a calibration curve prepared by measuring turbidity over time with a visible light photometer. Next, 100 μL aliquots of a polymer solution having twice as high as a concentration of each est well prepared using sterile ultrapure water were added to the wells of a 96-well plate. Subsequently, 100 μ1 aliquots of the Escherichia coli suspension were added to each well and mixed with the polymer solution, followed by culturing at 37° C. and measuring absorbance with a plate reader at 0, 2, 4, 6, 8 and 24 hours at OD 595. The final polymer test concentrations were made to be 4, 8, 16, 32, 64, 125, 250 and 500 mg/L. Escherichia growth at each concentration was similarly evaluated based on absorbance (turbidity) using control samples in the form of polyethylene glycol (PEG, Mn=5,000), polyethyleneimine (PEI, Mn=70,000) and penicillin/streptomycin (P/S, penicillin: 10,000 U, streptomycin: 10 mg/mL). The sample concentration that inhibited Escherichia coli growth after culturing for 24 hours was defined as the minimum inhibitory concentration (MIC).

### Example 19

### Confirmation of Lysis (SEM Observation)

100 μL, aliquots of a 64 mg/L polymer solution and 100 μL aliquots of Escherichia coli that were adjusted the value of OD₆₀₀ at 0.2 were added to a 96-well plate followed by culturing for 24 hours at 37° C. after mixing with the polymer solution. The final polymer test concentration was made to be 32 mg/L. Supernatant was then removed by centrifuging the plate for 10 minutes at 4000 rpm. PBS(−) was then added followed by centrifuging for 10 minutes at 4000 rpm and removing the supernatant. After additionally carrying out this procedure two times, 1% glutaraldehyde was added followed by allowing to stand undisturbed overnight in an incubator at 37° C. (fixation). Following fixation, the 1% glutaraldehyde was removed followed by washing with ultrapure water and sequentially drying with 35%, 50%, 75%, 90%, 95% and 100% ethanol in water. After drying overnight at room temperature, the bottom of the plate was cut out and subjected to ion coating with platinum-palladium. The plate was then immobilized on an SEM stand with carbon tape followed by observation of morphology with a field emission scanning electron microscope (JEPL, JSM-7600FA).

### Example 20

### Hemolysis Test

4.5 mL of human blood were collected into a vacuum blood collection tube containing 0.5 mL of 3.2% sodium citrate solution followed by centrifuging for 10 minutes at 2500 g to separate erythrocytes. 1 mL of the resulting erythrocyte solution was mixed with 9 mL of phosphate-buffered saline (PBS) followed by centrifuging for 5 minutes at 2500 g and removing about 9 mL of the supernatant. This washing procedure was additionally repeated twice followed by diluting the remaining erythrocyte solution with PBS to prepare a 4% human erythrocyte suspension.

100 μL aliquots of the 4% human erythrocyte suspension and 100 μ1 aliquots of each concentration of polymer solution were added to a 96-well plate followed by allowing to stand undisturbed for 1 hour at 37° C. The final polymer concentrations were made to be 3000, 2500, 2000, 1500, 1000, 500, 100 and 50 μg/mL. Subsequently, each polymer-treated solution was centrifuged for 5 minutes at 1000 g followed by transferring 100 μL aliquots of the supernatant to a different 96-well plate and evaluating the amount of hemoglobin released based on absorbance measured at 576 nm with a microplate reader. The 4% human erythrocyte suspension was used as a negative control, hemolysis (%) was defined as {(absorbance of polymer-treated solution)−(absorbance of PBS)}/{(absorbance of solution treated with Triton X-100) (absorbance of PBS)} based on a value of 100% (positive control) for a sample hemolyzed with Triton X-100.

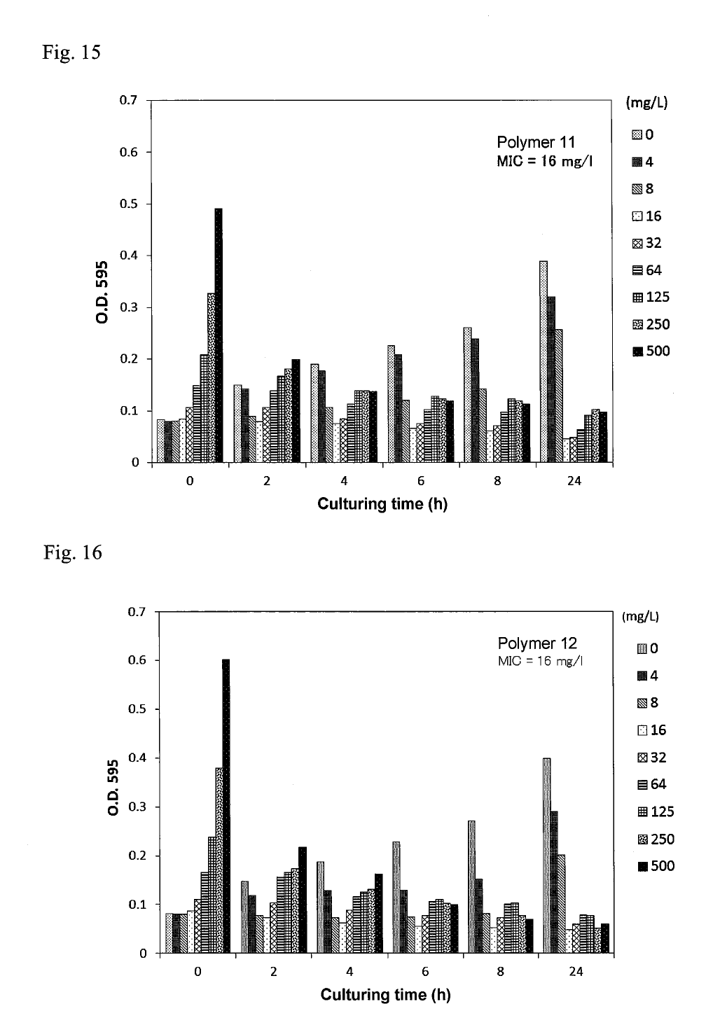

The results of the antibacterial activity test carried out in Example 18 are shown in FIGS. 15 to 18. Polymer 11 shown in FIG. 15 (Example 11: Homopolymer having polycarbonate for the main chain thereof and having only a side chain containing cationic groups) was able to inhibit the growth of Escherichia coli at an MIC value of about 16 mg, and was indicated to have antibacterial activity. On the other hand, Polymer 12 shown in FIG. 16 (Example 13: Copolymer having polycarbonate for the main chain thereof and containing side chains containing a cationic group and side chains expected to demonstrate biocompatibility at a ratio of 1:1) demonstrated an MIC value of about 16 mg despite a decrease in the density of side chains containing a cationic group in comparison with Polymer 11, and was indicated to have antibacterial activity against Escherichia coli that was equal to that of Polymer 11.

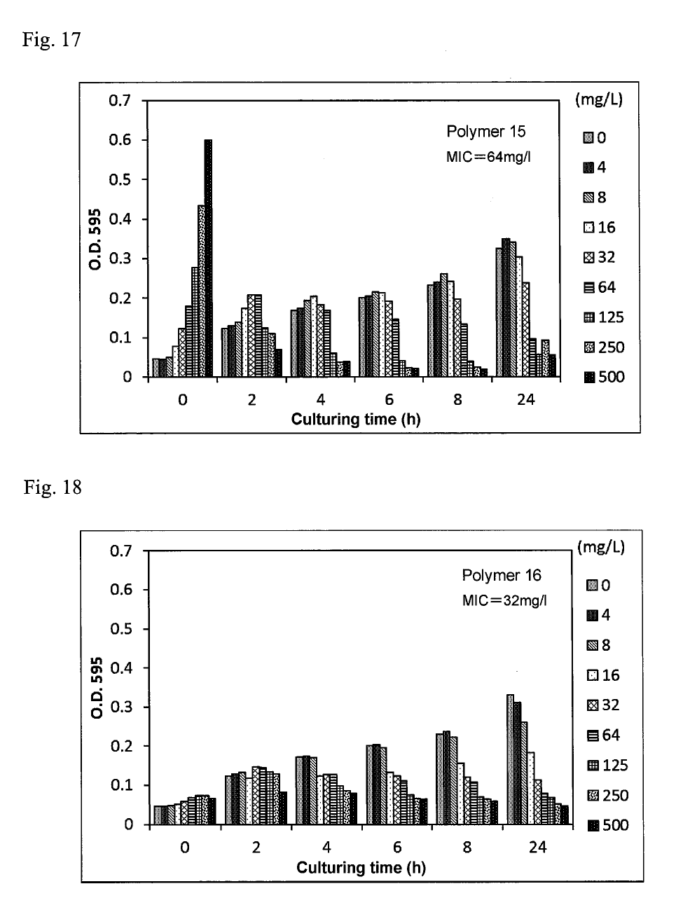

In addition, in the case of polymers having an acrylate structure for the main chain thereof, in contrast to Polymer 15 shown in FIG. 17 (Example 15: homopolymer having acrylate for the main chain and having only side chains containing a cationic group) demonstrating antibacterial activity against Escherichia coli at an MIC value of about 64 mg, Polymer 16 shown in FIG. 18 (Example 17: copolymer having acrylate for the main chain thereof and containing side chains containing a cationic group and side chains expected to demonstrate biocompatibility at a ratio of 1:1) demonstrated antibacterial activity at an MIC value of about 32 mg.

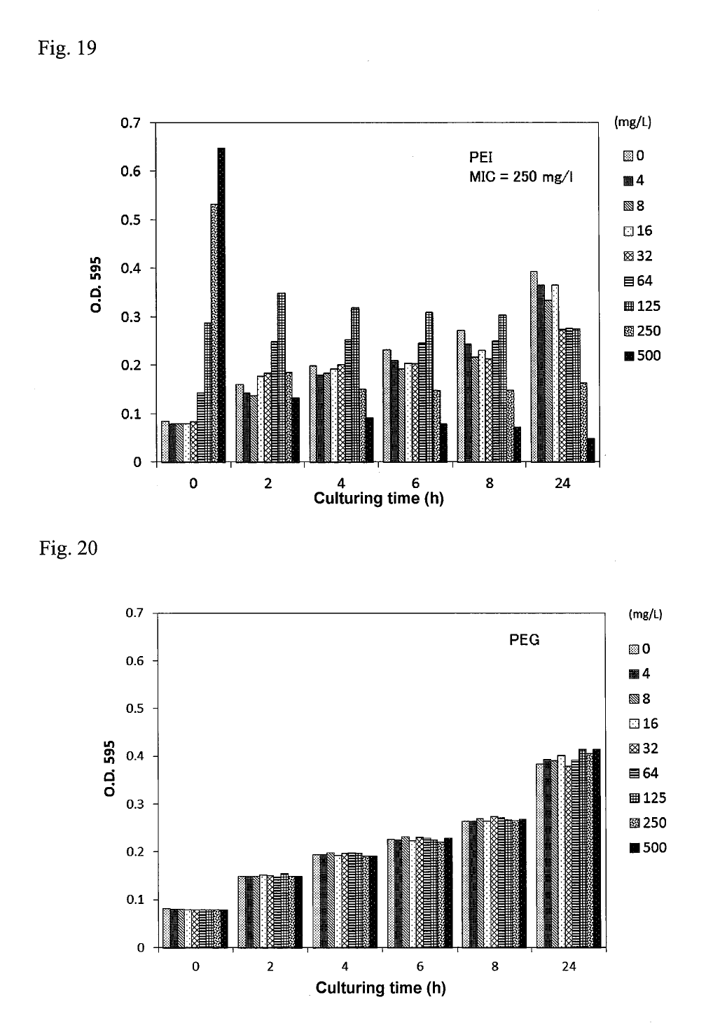

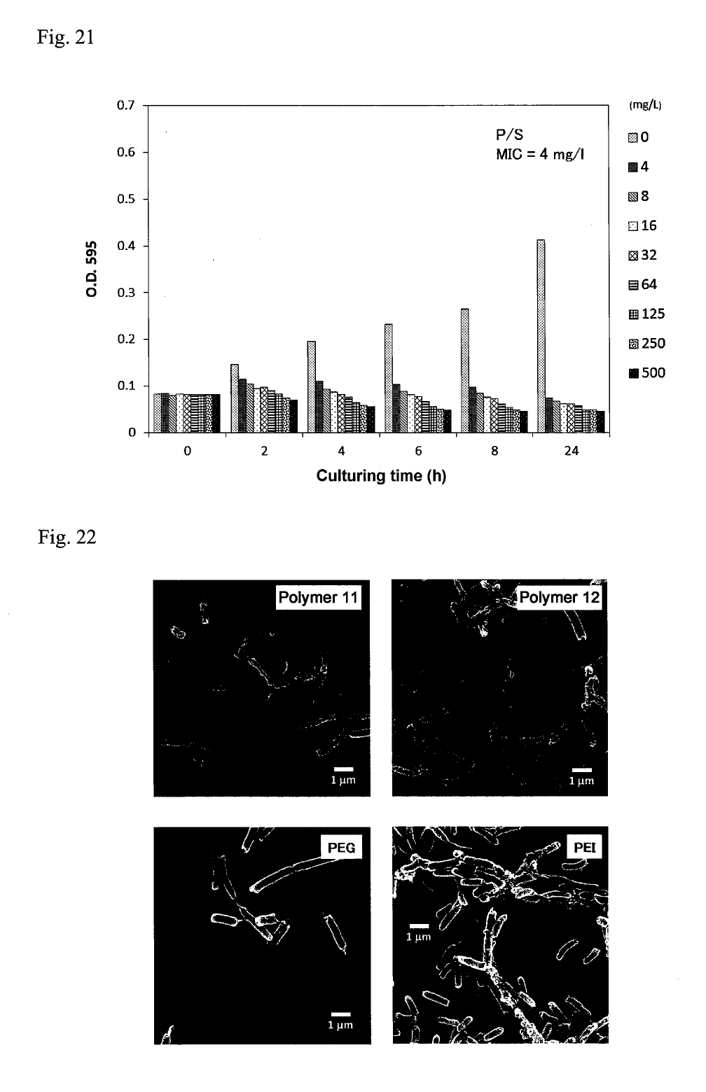

Based on the aforementioned results, Example 18 demonstrates that favorable antibacterial activity is observed for conventionally known polymers having side chains containing a cationic group, and that antibacterial action was observed to be more favorable than polyethyleneimine (PEI, FIG. 19) that is known to be an antibacterial material. In contrast, in the case of copolymers introduced with side chains expected to demonstrate biocompatibility in addition to side chains containing a cationic group, antibacterial activity equal to or better than that of homopolymers was shown to be observed in Example 18. This result is thought to indicate that, in comparison with a typical material that demonstrates biocompatibility in the form of polyethylene glycol (PEG, FIG. 20) not demonstrating any antibacterial activity whatsoever, introduction of side chains expected to demonstrate biocompatibility into a polymer does not significantly inhibit antibacterial activity attributable to the cationic group.

In addition, as shown in FIG. 22, in both the case of a homopolymer having side chains containing a cationic group (Polymer 11) and a copolymer containing side chains containing a cationic group and side chains expected to demonstrate biocompatibility at a ratio of 1:1 (Polymer 12), Escherichia coli was shown to be eradicated in a form such that the cell membrane thereof underwent degeneration, and a change in the specific form of that antibacterial action is thought to not occur due to the presence of side chains expected to demonstrate biocompatibility.

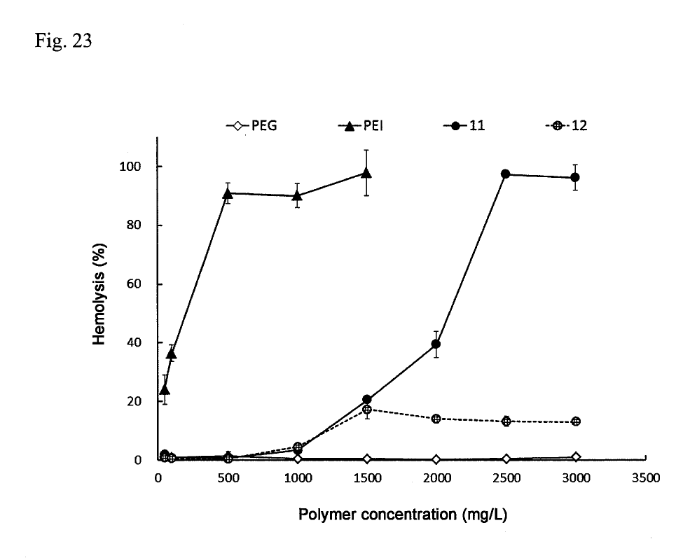

On the other hand, FIG. 23 shows the results of the hemolysis test carried out in Example 20. As is clear from FIG. 23, in the case of a conventional antibacterial material in the form of polyethyleneimine (PEI), although remarkable hemolysis occurs in the vicinity of the MIC value (250 mg), since hardly any hemolysis occurs at the concentrations required to inhibit growth of Escherichia coli (MIC value: about 16 mg) for both a copolymer according to the present invention having side chains containing a cationic group (Polymer 11) and a copolymer according to the present invention containing side chains containing a cationic group and side chains expected to demonstrate biocompatibility at a ratio of 1:1 (Polymer 12), both polymers were clearly determined to demonstrate favorable antibacterial activity within a range over which they demonstrate adequate biocompatibility. Moreover, in the case of Polymer 12 introduced with side chains expected to demonstrate biocompatibility, hemolysis was inhibited over a remarkably high concentration range of 1500 mg/L or more, thereby indicating that this polymer has adequate biocompatibility in the case of treating at a high concentration.

### INDUSTRIAL APPLICABILITY

The polymer of the present invention demonstrates superior antibacterial action at low concentrations while also being compatible with the body by inhibiting hemolysis and the like to a low level, thereby making it useful in applications in which it is used in contact with the body. Moreover, since hemolysis, biodegradation and other properties of the polymer of the present invention can be adjusted, it can be preferably used as an antibacterial agent and the like capable of serving as an alternative to the materials or coating materials of medical devices that may contact blood or other body components or as an alternative to antibiotics, thereby making it extremely industrially important.

1. A polymer constituted with a main chain and side chain moieties at least containing structures specified by the following (A) and (B) which are linked via a linker:
(A) a structure containing a cationic group; and,
(B) a structure expected to express biocompatibility.

2. The polymer according to claim 1, wherein the cationic group is a primary to quaternary ammonium group.

3. The according to claim 1, wherein the cationic group is a primary ammonium group.

4. The polymer according to claim 1, wherein the structure expected to express biocompatibility contains at least one ether structure.

5. The polymer according to claim 4, wherein the ether structure is a linear ether structure or cyclic ether structure.

6. The polymer according to claim 1, wherein the main chain is a biodegradable polymer, a non-biodegradable polymer or a copolymer thereof.

7. The polymer according to claim 1, wherein the main chain is a biodegradable polymer.

8. The polymer according to claim 1, which has a side chain moiety containing a primary ammonium group linked via a linker, and a side chain moiety containing a chain ether structure linked via a linker.

9. The polymer according to claim 1, wherein the linker is an ester bond.

10. The polymer according to claim 1, wherein the main chain is a polycarbonate chain or hydrocarbon chain.

11. The polymer according to claim 1, which is used in contact with biological tissue or blood.

12. A method for producing a polymer, comprising a step for mixing and ring-opening polymerization at least two types of monomer compounds selected from cyclic monomers represented by general formula (I): 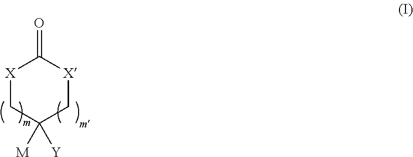
wherein
X and X′ mutually and independently represent —O—, —NH— or —CH₂— provided that at least one thereof is not —CH₂—,
Y represents a group represented by -L-Z (wherein, Z represents a side chain moiety having a cationic group, a side chain moiety Z having a group serving as a precursor of a cationic group, or a side chain moiety Z² capable of retaining intermediate water in the body, and L represents a linker between the main chain and Z that is selected from unit structures having an alkylene group, ether bond, thioether bond, ester bond, amide bond, urethane bond, urea bond or a combination thereof),
M represents a hydrogen atom or a linear or branched alkyl group having 3 carbon atoms or less, and
m and m′ mutually and independently represent an integer of 0 to 5, provided that at least one of m and m′ is not zero when X and X′ are both —O—, and the sum of m and
m′ is 7 or less); wherein, a first cyclic monomer has Z¹ and a second cyclic monomer has Z².

13. The method for producing a polymer according to claim 12, wherein the mixing ratio between the first cyclic monomer and the second cyclic monomer is 1:99 to 99:1 in terms of the molar ratio thereof.

14. A medical device having the polymer according to claim 1 on at least a portion of the surface thereof.

15. An antibacterial agent comprising the polymer according to claim 1.
